(19) United States
(12) Patent Application Publication
(10) Pub. No.: US 2023/0220053 A1
(43) Pub. Date: Jul. 13, 2023

(54) ANTI-SARS-CoV-2 ANTIBODIES AND USES THEREOF

(71) Applicant: Elpis Biopharmaceuticals, Lexington, MA (US)

(72) Inventors: Yan CHEN, Lexington, MA (US); Kehao ZHAO, Lexington, MA (US); Jenna NGUYEN, Lexington, MA (US); Ning JIANG, Lexington, MA (US)

(73) Assignee: Elpis Biopharmaceuticals, Lexington, MA (US)

(21) Appl. No.: 17/925,967

(22) PCT Filed: May 17, 2021
(86) PCT No.: PCT/US2021/032747

§ 371 Date: Nov. 17, 2022

Related U.S. Application Data
(60) Provisional application No. 63/026,486, filed on May 18, 2020.

(51) Int. Cl. C07K 16/10; G01N 33/569

(52) U.S. Cl. CPC C07K 16/10; G01N 33/56983; C07K 2317/21; C07K 2317/76; C07K 2317/92; C07K 2317/622; G01N 2333/165; G01N 2469/10; G01N 2800/26

(57) ABSTRACT

Disclosed herein are anti-SARS-CoV-2 spike protein antibodies and methods of using such for therapeutic and/or diagnostic purposes. Also provided herein are methods for producing such antibodies.

### CROSS REFERENCE TO RELATED APPLICATION

This application claims benefit under 35 U.S.C. § 119(e) of the U.S. provisional application No. 63/026,486 filed May 18, 2020, the content of which is incorporated by reference herein in its entirety.

### BACKGROUND OF THE INVENTION

Severe respiratory syndrome coronavirus-2 (SARS-CoV-2), a novel coronavirus, causes Coronavirus Disease 2019 (COVID-19), which poses a severe, worldwide health risk. Its genome encodes at least 27 proteins, including 15 nonstructural proteins, 4 structural proteins, and 8 auxiliary proteins. Spike glycoprotein (S), a structural protein located on the outer envelope of the virion, binds to the human receptor angiotensin-converting enzyme 2 (ACE2). The S glycoprotein of SARS-CoV, MERS-CoV, and SARS-CoV-2 has 1104 to 1273 amino acids and contains an amino (N)-terminal S1 subunit and a carboxyl (C)-terminal S2 subunit. In the S1 subunit, the receptor-binding domain (RBD), spanning about 200 residues, consists of two subdomains: the core and external subdomains. The RBD core subdomain is responsible for the formation of S trimer particles. The first step in viral entry is thought to be the binding of the viral trimeric spike protein to ACE2.

There is a need in the art to identify effective treatments against SARS-CoV-2 infections and COVID-19. In particular, there is a need to develop antibodies capable of interfering with the ability of SARS-CoV-2 to bind to target cells, particularly to ACE2.

### SUMMARY OF THE INVENTION

The present disclosure is based, at least in part, on the development of superior anti-SARS-CoV-2 antibodies (a.k.a., anti-S1 antibodies) having high binding affinity and specificity to a S1 subunit of a SARS-CoV-2 spike protein S. In some cases, the anti-S1 antibody binds to a receptor binding domain (RBD) of S1. In other examples, the anti-S1 antibody binds to a region outside a RBD of S1. The anti-S1 antibodies disclosed herein showed ability to block the binding of S1 (e.g., the RBD) to angiotensin-converting enzyme 2 (ACE2), which in turn may inhibit the ability of SARS-CoV-2 to effectively infect cells. Accordingly, the antiSARS-CoV2 antibodies disclosed here are expected to be effective in blocking entry of SARS-CoV2 in to host cells, thereby inhibiting SARS-CoV2 infection of hosts such as human subjects.

Accordingly, the present disclosure provides, in some aspect, an isolated antibody that binds the S1 protein, wherein the antibody binds to the same epitope as a reference antibody or competes against the reference antibody from binding to S1. The reference antibody may be 2020EP53-D06, 2020EP54-H01, 2020EP54-E12, 2020EP54-B12, 2020EP54-B02, 2020EP54-E10, 2020EP60-F05, 2020EP60-A12, 2020EP61-A08, 2020EP61-C12, 2020EP64-G10, 2020EP66-D03, 2020EP64-C08, 2020EP66-A07, 2020EP71-E04, or 2020EP75-E02.

In some embodiments the anti-S1 antibody may comprise: (a) a heavy chain complementary determining region 1 (HC CDR1), a heavy chain complementary determining region 2 (HC CDR2), and a heavy chain complementary determining region 3 (HC CDR3), wherein the HC CDR1, HC CDR2, and HC CDR3 collectively are at least 80% identical to the heavy chain CDRs of an antibody disclosed herein; and/or (b) a light chain complementary determining region 1 (LC CDR1), a light chain complementary determining region 2 (LC CDR2), and a light chain complementary determining region 3 (LC CDR3), wherein the LC CDR1, LC CDR2, and LC CDR3 collectively are at least 80% identical to the light chain CDRs of an antibody disclosed herein.

In some embodiments, the anti-S1 antibody disclosed herein may collectively contain no more than 8 amino acid residue variations as compared with the HC CDRs of the reference antibody; and/or wherein the LC CDRs of the antibody collectively contain no more than 8 amino acid residue variations as compared with the LC CDRs of the reference antibody.

In some embodiments, the anti-S1 antibody disclosed herein may comprise a V_{H} that is at least 85% identical to the V_{H} of the reference antibody, and/or a V_{L} that is at least 85% identical to the V_{L} of the reference antibody. The isolated antibody may have a binding affinity of less than 10 mM to S1. In some examples, the anti-S1 antibody may comprise the same heavy chain complementary determining regions (HC CDRs) and the same light chain complementary determining regions (LC CDRs) as the reference antibody. In specific examples, the anti-S1 antibody may comprise the same V_{H} and the same V_{L} as the reference antibody.

Any of the anti-S1 antibodies disclosed herein can be a human antibody or a humanized antibody. The antibody may be a full-length antibody or an antigen-binding fragment thereof. In some instances, the antibody can be a full-length antibody, which can be an IgG1 molecule. Alternatively, the antibody may be a single-chain antibody (scFv).

In another aspect, the present disclosure provides a nucleic acid or a set of nucleic acids, which collectively encodes any of the anti-S1 antibodies disclosed herein. In some embodiments, the nucleic acid or the set of nucleic acids can be a vector or a set of vectors, for example, expression vectors. Also provided herein are host cells comprising any of the nucleic acids or the sets of nucleic acids disclosed herein, as well as pharmaceutical compositions comprising any of the anti-S1 antibodies disclosed herein, any of the encoding nucleic acids or sets of nucleic acids, or host cells comprising such, and a pharmaceutically acceptable carrier.

In yet another aspect, the present disclosure provides a method of treating or inhibiting a coronavirus infection in a subject. The subject may be in need of such treating, preventing, or inhibiting. The coronavirus may be severe acute respiratory syndrome coronavirus 2 (SARS-CoV-2), severe acute respiratory syndrome coronavirus (SARS-CoV), or Middle East respiratory syndrome coronavirus (MERS-CoV). The subject may have, may be suspected of having, or may be at risk of having, a disease associated with a coronavirus infection. The disease may be COVID-19, SARS, or MERS. In a particular aspect, the coronavirus may be SARS-CoV-2. In one example, the disease may be COVID-19. The subject may be a human patient.

The method may comprise administering to the subject in need thereof an effective amount of any of the anti-S1 antibodies disclosed herein, the encoding nucleic acids, or the pharmaceutical composition comprising such. Also within the scope of the present disclosure are pharmaceutical compositions as disclosed herein for use in treating a disease caused by a coronavirus, such as those described herein, as well as use of any of the anti-S1 antibodies disclosed herein for manufacturing a medicament for use in treating any of the target diseases as also disclosed herein.

Further, the present disclosure provides a method of detecting presence of SARS-CoV-2), comprising: (i) contacting an antibody of any one of claims 1-12 with a sample suspected of containing S1 subunit or a fragment comprising the RBD domain of SARS-CoV-2, and (ii) detecting binding of the antibody to RBD and/or S1. The antibody may be conjugated to a detectable label. In some embodiments, the contacting step may be performed by administering the antibody to a subject. In other embodiments, the sample may be a biological sample obtained from a subject (e.g., a human patient suspected of having a coronavirus infection, such as a SARS-CoV2 infection), for example, a blood sample.

The present disclosure also provides a method of producing an antibody binding to S1, including the RBD, comprising: (i) culturing the host cell disclosed herein under conditions allowing for expression of the antibody that binds the RBD and/or S1; and (ii) harvesting the antibody thus produced from the cell culture.

Also within the present disclosure are pharmaceutical compositions for use in inhibiting or treating a coronavirus infection or a disease or condition associated with a coronavirus infection, the composition comprising any of the antiSARS-CoV2 antibodies or coding nucleic acid(s) thereof and a pharmaceutically acceptable carrier, and uses of such antibodies or encoding nucleic acids for manufacturing a medicament for use in inhibiting a coronavirus infection and/or treating a disease or condition associated with a coronavirus infection.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein.

FIG. 1 shows the results of single point screening ELISA for single-chain (scFv) binders of the RBD of SARS-CoV-2 spike protein S1.

FIGS. 2A-2B show binding of the RBD (FIG. 2A) and S1 protein (FIG. 2B) to ACE2 in ELISA tests.

FIG. 3 show binding activity of S1 protein to ACE2 in ACE2-expressing CHO-K1 cells.

FIG. 4 shows SDS-PAGE gels indicating the purity of purified anti-S1 antibodies.

FIGS. 5A-5B show binding of anti-S1 scFvs to the RBD (FIG. 5A) or to S1 (FIG. 5B).

FIGS. 6A-6B show sensor gram examples of 2020EP054-E10 binding to the RBD (FIG. 6A) and S1 protein (FIG. 6B), respectively

FIGS. 7A-7B show the neutralizing activities of anti-S1 scFvs in blocking the binding of 0.2 nM RBD (FIG. 7A) or 2 nM S1 (FIG. 7B) to ACE2 using ELISA.

FIG. 8 shows the neutralizing activity of anti-S1 scFvs in blocking the binding of 18 nM S1 to ACE2 in CHO-K1 cells, as measured by FACS.

FIGS. 9A-9D show binding of IgG1 antibodies to the RBD (FIGS. 9A and C) and S1 (FIGS. 8B and D) using ELISA.

FIGS. 10A-10B show examples of sensor grams of IgG antibodies (2020EP54-E10 IgG) to RBD (FIG. 10A) and S1 (FIG. 10B) protein in SPR, respectively.

FIG. 11 shows binding activities of IgG antibodies to S protein in ELISA.

FIGS. 12A-12B show binding of IgG1 antibodies to the RBD (FIG. 12A) and S1 (FIG. 12B) of SARS-COV using ELISA.

FIGS. 13A-13H show the SPR sensor gram examples of 2020EP054-E10 (FIGS. 13A-D) and 2020EP054-B12 (FIGS. 13E-H) binding to the RBD and S1 protein of SARS-COV-2 and SARS-COV, respectively.

FIG. 14 shows examples of IgG antibody neutralization of S1 protein binding to ACE2 in ELISA.

FIG. 15 shows examples of IgG antibody neutralization of S1 protein binding to human ACE2/CHOK1 cells in a FACS assay.

### DETAILED DESCRIPTION OF THE INVENTION

Provided herein are antibodies capable of binding to a S1 subunit of a SARS-CoV-2 spike protein S (“anti-S1 antibodies”). In some examples, the anti-S1 antibodies more particularly bind to the receptor binding domain (“RBD”) of S1 (“anti-RBD antibodies”). The term “anti-S1 antibodies” includes anti-RBD antibodies. The anti-S1 antibodies disclosed herein show high binding affinity to S1, and in some examples, high binding affinity to RBD. In some embodiments, the anti-S1 antibodies block the ability of S1, and in some cases the RBD, to bind ACE2. Anti-S1 antibodies may also inhibit the ability of SARS-CoV-2 virus to bind to ACE2, thereby inhibiting the virus’ ability to infect cells. In some examples, anti-S1 antibodies described herein may not bind to the RBD, but are still capable of neutralizing a viral infection through an allosteric mechanism. Such antibodies may show synergistic effects in combination with other antibodies that directly bind to the RBD, which may be important in engaging host cells.

S1 is a subunit of the SARS-CoV-2 spike protein S as disclosed herein. The S1 polypeptide is known in the art. The RBD in S1 is also known in the art. In one example, the sequence of the RBD can include amino acids 328-533 of S protein.

The spike protein of SARS-CoV-2 is thought to be essential for the ability of the virus to infect cells, specifically through binding to ACE2. Thus, the anti-S1 antibodies disclosed herein can serve as therapeutic agents for treating diseases associated with SARS-CoV-2, for example, COVID-19. In addition, the anti-S1 antibodies disclosed herein can serve as diagnostic agents for detecting presence of SARS-CoV-2, e.g., SARS-CoV-2-positive cells. The antibodies disclosed herein may also be used for research purposes.

### I. Antibodies Binding to S1 and the RBD Thereof

The present disclosure provides antibodies binding to the S1 subunit of SARS-CoV-2 spike protein S. In some embodiments, the anti-S1 antibodies disclosed herein are capable of binding to the RBD. As such, the antibodies disclosed herein may be used for either therapeutic or diagnostic purposes to prevent, treat or diagnose a SARS-CoV-2 infection, or COVID-19. As used herein, the term “anti-S1 antibody” refers to any antibody capable of binding to a S1 polypeptide, including a RBD polypeptide. The anti-S1 antibody may bind an epitope located with the RBD, or may bind an epitope located outside the RBD.

An antibody (interchangeably used in plural form) is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody”, e.g., an anti-S1 antibody, including an anti-RBD antibody, encompasses not only intact (e.g., full-length) polyclonal or monoclonal antibodies, but also antigen-binding fragments thereof (such as Fab, Fab′, F(ab′)2, Fv), single-chain antibody (scFv), fusion proteins comprising an antibody portion, humanized antibodies, chimeric antibodies, diabodies, single domain antibody (e.g., nanobody), single domain antibodies (e.g., a V_{H} only antibody), multispecific antibodies (e.g., bispecific antibodies) and any other modified configuration of the immunoglobulin molecule that comprises an antigen recognition site of the required specificity, including glycosylation variants of antibodies, amino acid sequence variants of antibodies, and covalently modified antibodies. An antibody, e.g., anti-Galectin-9 antibody, includes an antibody of any class, such as IgD, IgE, IgG, IgA, or IgM (or subclass thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

A typical antibody molecule comprises a heavy chain variable region (V_{H}) and a light chain variable region (V_{L}), which are usually involved in antigen binding. The V_{H} and V_{L} regions can be further subdivided into regions of hypervariability, also known as “complementarity determining regions” (“CDR”), interspersed with regions that are more conserved, which are known as “framework regions” (“FR”). Each V_{H} and V_{L} is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the Chothia definition, the AbM definition, and/or the contact definition, all of which are well known in the art. See, e.g., Kabat, E.A., et al. (1991) Sequences of Proteins of Immunological Interest, Fifth Edition, U.S. Department of Health and Human Services, NIH Publication No. 91-3242, Chothia et al., (1989) Nature 342:877; Chothia, C. et al. (1987) J. Mol. Biol. 196:901-917, Al-lazikani et al (1997) J. Molec. Biol. 273:927-948; and Almagro, J. Mol. Recognit. 17:132-143 (2004). See also hgmp.mrc.ac.uk and bioinf.org.uk/abs).

The anti-S1 antibody described herein may be a full-length antibody, which contains two heavy chains and two light chains, each including a variable domain and a constant domain. Alternatively, the anti-S1 antibody can be an antigen-binding fragment of a full-length antibody. Examples of binding fragments encompassed within the term “antigen-binding fragment” of a full length antibody include (i) a Fab fragment, a monovalent fragment consisting of the V_{L}, V_{H}, C_{L} and C_{H}1 domains; (ii) a F(ab′)₂ fragment, a bivalent fragment including two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR) that retains functionality. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules known as single chain Fv (scFv). See e.g., Bird et al. (1988) Science 242:423-426; and Huston et al. (1988) Proc. Natl. Acad. Sci. USA 85:5879-5883.

The antibodies described herein can be of a suitable origin, for example, murine, rat, or human. Such antibodies are non-naturally occurring, i.e., would not be produced in an animal without human act (e.g., immunizing such an animal with a desired antigen or fragment thereof or isolated from antibody libraries). Any of the antibodies described herein, e.g., anti-S1 antibody, can be either monoclonal or polyclonal. A “monoclonal antibody” refers to a homogenous antibody population and a “polyclonal antibody” refers to a heterogeneous antibody population. These two terms do not limit the source of an antibody or the manner in which it is made.

In some embodiments, the anti-S1 antibodies are human antibodies, which may be isolated from a human antibody library or generated in transgenic mice. For example, fully human antibodies can be obtained by using commercially available mice that have been engineered to express specific human immunoglobulin proteins. Transgenic animals that are designed to produce a more desirable (e.g., fully human antibodies) or more robust immune response may also be used for generation of humanized or human antibodies. Examples of such technology are Xenomouse™ from Amgen, Inc. (Fremont, Calif.) and HuMAb-Mouse™ and TC Mouse™ from Medarex, Inc. (Princeton, N.J.). In another alternative, antibodies may be made recombinantly by phage display or yeast technology. See, for example, U.S. Pat. Nos. 5,565,332; 5,580,717; 5,733,743; and 6,265,150; and Winter et al., (1994) Annu. Rev. Immunol. 12:433-455. Alternatively, the antibody library display technology, such as phage, yeast display, mammalian cell display, or mRNA display technology as known in the art can be used to produce human antibodies and antibody fragments in vitro, from immunoglobulin variable (V) domain gene repertoires from unimmunized donors.

In other embodiments, the anti-S1 antibodies may be humanized antibodies or chimeric antibodies. Humanized antibodies refer to forms of non-human (e.g., murine) antibodies that are specific chimeric immunoglobulins, immunoglobulin chains, or antigen-binding fragments thereof that contain minimal sequence derived from non-human immunoglobulin. In general, humanized antibodies are human immunoglobulins (recipient antibody) in which residues from a CDR of the recipient are replaced by residues from a CDR of a non-human species (donor antibody) such as mouse, rat, or rabbit having the desired specificity, affinity, and capacity. In some instances, one or more Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In some instances, the humanized antibody may comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Antibodies may have Fc regions modified as described in WO 99/58572. Other forms of humanized antibodies have one or more CDRs (one, two, three, four, five, or six) which are altered with respect to the original antibody, which are also termed one or more CDRs “derived from” one or more CDRs from the original antibody. Humanized antibodies may also involve affinity maturation. Methods for constructing humanized antibodies are also well known in the art. See, e.g., Queen et al., Proc. Natl. Acad. Sci. USA, 86:10029-10033 (1989).

In some embodiments, the anti-S1 antibody disclosed herein can be a chimeric antibody. Chimeric antibodies refer to antibodies having a variable region or part of variable region from a first species and a constant region from a second species. Typically, in these chimeric antibodies, the variable region of both light and heavy chains mimics the variable regions of antibodies derived from one species of mammals (e.g., a non-human mammal such as mouse, rabbit, and rat), while the constant portions are homologous to the sequences in antibodies derived from another mammal such as human. In some embodiments, amino acid modifications can be made in the variable region and/or the constant region. Techniques developed for the production of “chimeric antibodies” are well known in the art. See, e.g., Morrison et al. (1984) Proc. Natl. Acad. Sci. USA 81, 6851; Neuberger et al. (1984) Nature 312, 604; and Takeda et al. (1984) Nature 314:452.

In some embodiments, the anti-S1 antibodies described herein specifically bind to the corresponding target protein (e.g., S1 or RBD) or an epitope thereof. An antibody that “specifically binds” to a protein or an epitope is a term well understood in the art. A molecule is said to exhibit “specific binding” if it reacts more frequently, more rapidly, with greater duration and/or with greater affinity with a particular target protein than it does with alternative targets. An antibody “specifically binds” to a target protein or epitope if it binds with greater affinity, avidity, more readily, and/or with greater duration than it binds to other substances. For example, an antibody that specifically (or preferentially) binds to a protein or an antigenic epitope therein is an antibody that binds this target protein with greater affinity, avidity, more readily, and/or with greater duration than it binds to other proteins or other epitopes in the same antigen. It is also understood with this definition that, for example, an antibody that specifically binds to a first target protein may or may not specifically or preferentially bind to a second target protein. As such, “specific binding” or “preferential binding” does not necessarily require (although it can include) exclusive binding. In some examples, an antibody that “specifically binds” to a target protein or an epitope thereof may not bind to other antigens or other epitopes in the same protein (i.e.., only baseline binding activity can be detected in a conventional method).

In some embodiments, an anti-S1 antibody as described herein has a suitable binding affinity for the target protein (e.g., S1 or RBD) or antigenic epitopes thereof. As used herein, “binding affinity” refers to the apparent association constant or K_{A}. The K_{A} is the reciprocal of the dissociation constant (K_{D}). The anti-S1 antibody described herein may have a binding affinity (K_{D}) of at least 100 nM, 10 nM, 1 nM, 0.1 nM, or lower for S1 or RBD. An increased binding affinity corresponds to a decreased K_{D}. Higher affinity binding of an antibody for a first antigen relative to a second antigen can be indicated by a higher K_{A} (or a smaller numerical value K_{D}) for binding the first antigen than the K_{A} (or numerical value K_{D}) for binding the second antigen. In such cases, the antibody has specificity for the first antigen (e.g., a first protein in a first conformation or mimic thereof) relative to the second antigen (e.g., the same first protein in a second conformation or mimic thereof; or a second protein). Differences in binding affinity (e.g., for specificity or other comparisons) can be at least 1.5, 2, 3, 4, 5, 10, 15, 20, 37.5, 50, 70, 80, 90, 100, 500, 1000, 10,000 or 10⁵ fold. In some embodiments, any of the anti-S1 antibodies may be further affinity matured to increase the binding affinity of the antibody to the target antigen or antigenic epitope thereof.

Binding affinity (or binding specificity) can be determined by a variety of methods including equilibrium dialysis, equilibrium binding, gel filtration, ELISA, surface plasmon resonance, or spectroscopy (e.g., using a fluorescence assay). Exemplary conditions for evaluating binding affinity are in HBS-P buffer (10 mM HEPES pH7.4, 150 mM NaCl, 0.005% (v/v) Surfactant P20). These techniques can be used to measure the concentration of bound binding protein as a function of target protein concentration. The concentration of bound binding protein ([Bound]) is generally related to the concentration of free target protein ([Free]) by the following equation:

$$\left[\text{Bound}\right] = \left[\text{Free}\right] / \left(\text{Kd+} \left[\text{Free}\right]\right)$$

It is not always necessary to make an exact determination of K_{A}, though, since sometimes it is sufficient to obtain a quantitative measurement of affinity, e.g., determined using a method such as ELISA or FACS analysis, is proportional to K_{A}, and thus can be used for comparisons, such as determining whether a higher affinity is, e.g., 2-fold higher, to obtain a qualitative measurement of affinity, or to obtain an inference of affinity, e.g., by activity in a functional assay, e.g., an in vitro or in vivo assay.

In some embodiments, the anti-S1 antibody disclosed herein has an EC₅₀ value of lower than 10 nM, e.g., < 1 nM, < 0.5 nM, or lower than 0.1 nM, for binding to S1 or the RBD. As used herein, EC₅₀ values refer to the minimum concentration of an antibody required to bind to 50% of the S1 or the RBD provided in a binding assay. IC₅₀ may also refer to the concentration of an antibody required to block 50% of S1 or the RBD from binding to a human ACE2-expressing cell population. EC₅₀ values can be determined using conventional assays and/or assays disclosed herein. See, e.g., Examples below.

A number of exemplary anti-S1 antibodies are provided below (CDRs indicated in bold as determined by the Chothia approach (Chothia et al. (1992) J. Mol. Biol., 227, 776-798, Tomlinson et al. (1995) EMBO J., 14, 4628-4638 and Williams et al.(1996) J. Mol. Biol., 264, 220-232). See also the website for the alignment tool of the variable sequences of antibodies (vbase) at the MRC Laboratory of Molecular Biology (LMB) of Cambridge Biomedical Campus, Cambridge, UK.

>2020Ep53-d06
V_{H}
QVQLVQSGGGWQPGRSLRLSCAASGFTFSSYAMHWVRQAPGKGLEWVAVI
SYDGSNKYYADSVKGRFTISRDNSKNTLYLQMNSLRAEDTAVYYCARDGE
DGYNYISPFDYWGQGTLVTVSS (SEQ.ID.NO: 1)
V_{L}
SYELTQPPSVSVAPGETARITCRGNDIGSKSVHWYQQKPGQAPVLVLYYD
SDRPSGIPERFSGSNSGNTATLTISSVEAGDEADYYCQVWDIDVVFGGGT
KLTVL (SEQ.ID.NO: 2)

>2020Ep54-h01
V_{H}
QVQLVESGGGVVQPGRSLRLSCAASGFTFSSYGMHWVRQAPGKGLEWVAV
ISYDGSNKYYADSVKGRFTISRDNSKNTLYLQMNSLRAEDTAVYYCAKGR
GWYLDYWGQGTLVTVSS (SEQ.ID NO: 3)
V_{L}
NFMLTQPPSVSVSPGQTASITCSGDKLGDKYACWYQQKPGQSPVLVMYED
YKRPSGIPARFSGSNSGNTATLTISRVEGGDEGDYYCQVWDSRGPEVIFG
GGTKLTVL (SEQ.ID.NO:4)

>2020Ep54-e12
V_{H}
EVQLVESGGGLVQPGGSLRLSCAVTGFIVSSNYMNWVRQAPGKGLEWVSV
IYSGGTTYYADSVKGRFTISRDKSKNTLYLQMNNLRAEDTAMYYCATLGN
DYGDYGTDYWGQGTLVTVSS (SEQ.ID.NO:5)
V_{L}
QLVLTQPPSVSVSPGQTASISCSGDRLGQKYTSWYQQRPGQSPVLIMYQD
NKRPSGIPERFSGSNSGNTATLTISGIQSMDEADYYCQVWDSGSDHAVFG
GGTQLTVL (SEQ.ID.NO:6)

>2020Ep54-b12
V_{H}
EVQLVESGGGLVQPGGSLRLSCAASGFAFSSYAMSWVRQAPGKGLEWVSA
ISGSGGSTYYADSVKGRFTISRDNSKNTLYLQMNSLRAEDTAVYYCAKVT
RVGFDYWGQGTLVTVSS (SEQ.ID.NO:7)
V_{L}
SYELTQPPSVSVAPGKTARITCGGNNIGSKSVHWYQQKPGQAPVLVIYYD
SDRPSGIPERFSGSNSGNTATLTISRVEAEDEADYYCQVWDSSSDVVFGG
GTKLTVL (SEQ.ID.NO:8)

>2020Ep54-b02
V_{H}
QVQLVQSGAEVKKPGASVKVSCKASGYTFIGYYMHWMRQAPGQGLEWMGR
INPNSGGTNYAEKFQGRVTMTRDTSISTAYMELSRLTFNDTAVYYCARWD
SSSWKFDSWGQGTLVTVSS (SEQ.ID.NO:9)
V_{L}
EIVLTQSPSSLSASVGDRVTITCQASQDISNYLNWYQQKPGKAPKLLIYD
ASNLETGVPSRFSGSGSGTDFTFTISSLQPEDIATYYCQQYDNL
PITFGQGTRLEIK (SEQ.ID.NO:10)

>2020Ep54-e10
V_{H}
QVQLVQSGAEVRKPGSSVKVSCKASGGTFSSFAISWVRQAPGQGLEWMGG
IIPIFDTATYAQNFQGRVRMTADESTNTAYMELSSLTSEDTAVYYCARST
SYYDSRGDYKVGDFDYWGQGTLVTVSS
(SEQ.ID.NO:11)
V_{L}
AIRMTQSPSSLSASVGDRVTITCQASQDISNYLNWYQQKPGKAPKLLIYD
ASNLETGVPSRFSGSGSGTDFTFTISSLQPEDIATYYCQQYDNLPLTFGG
GTKVEIK (SEQ.ID.NO:12)

>2020Ep60-f05
V_{H}
QVQLVESGGGVVQPGGSLRLSCAASGFTFSSYGMHWVRQAPGKGLEWVAV
ISYDGSNKYYADSVKGRFTISRDNSKNTLYLQMNSLRAEDTAVYYCAKAR
VALGYFDWLLYDGMDVNGQGTTVTVSS
(SEQ.ID.NO:13)
V_{L}
SSELTQPPSVSVSPGQTASITCSGDKLGNKYVFWYQQKPGQSPILVIYQD
NRRPSGIPERFSGSNSGNTATLTISGDPAMDEADYYCQAWDSSTVVFGGG
TKLTVL (SEQ.ID.NO:14)

>2020Ep60-a12
V_{H}
EVQLVESGGGVVQPGRSLRLSCAASGFTFSSYGMHWVRQAPGKGLEWVAV
ISYDGSNKYYADSVKGRFTISRDNSKNTLYLQMNSLRAEDTAVYYCAKGL
EYYFDYWGQGTLVTVSS (SEQ.ID.NO:15)
V_{L}
SYELTQPPSLSVSPGQTARITCSGDALPEQYAYWYQQKPGQAPVLVIYKD
SERPSGIPERFSGSSSGTTVTLTISGVQAEDEADYYCQAWDSSTVVFGGG
TKLTVL (SEQ.ID.NO:16)

>2020Ep61-a08
V_{H}
QVQLVQSGAEVKKPGASVKVSCKASGYTFTSYYMHWVRQAPGQGLEWMGI
INPSGGSTSYAQKFQGRVTMTRDTSTSTVYMELSSLRSEDTAVYYCARES
GNYYYDSSGYTFDYWGQGTLVTVSS
(SEQ.ID.NO:17)
V_{L}
SYVLTQPLSVSVALGQTARITCGGNNIGSKNVHWYQQKPGQAPVLVIYYD
SDRPSGIPERFSGSNSGNTATLTISGTQAMDEADYYCQAWDSSTVVFGGG
TKLTVL (SEQ.ID.NO:18)

>2020Ep61-c12
V_{H}
EVQLVESGGGLVQPGGSLRLSCAASGFTFSSYAMSWVRQAPGKGLEWVSA
ISGSGGSTYYADSVKGRFTISRDNSKNTLYLQMNSLRAEDTAVYYCAKDE
YSYGSLYFDYWGQGTLVTVSS (SEQ.ID.NO:19)
V_{L}
SYELTQPPSVSVAPGQTARITCGGNNIGSKSVHWYQQKPGQAPVLWYDDS
DRPSGIPERFSGSNSGNTATLTISRVEAGDEADYYCQVWDSSSDSVVFGG
GTKLTVL (SEQ.ID.NO:20)

>2020Ep64-g10
V_{H}
QVQLVQSGGGWQPGRSLRLSCAASGFTFSSYAMHWVRQAPGKGLEWVAVI
SYDGSNKYYADSVKGRFTISRDNSKNTLYLQMNSLRAEDTAVYYCARDGE
DGYNYISPFDYWGQGTLVTVSS (SEQ.ID.NO:1)
V_{L}
SYELTQPPSVSVAPGETARITCRGNDIGSKSVHWYQQKPGQAPVLVLYYD
SDRPSGIPERFSGSNSGNTATLTISSVEAGDEADYYCHVWDIDVVFGGGT
KLTVL (SEQ. D.NO:21)

>2020Ep66-d03
V_{H}
QVQLQQWGAGLLKPSETLSLTCAVYGGSFSGYYWSWIRQPPGKGLEWIGE
INHSGSTNYNPSLKSRVTISVDTSKNQFSLKLSSVTAADTAVYYCARGPP
LWNYGKGFDYWGQGTLVTVSS (SEQ.ID.NO:22)
V_{L}
QPVLTQPPSASGTPGQRVTISCSGSSSNIGSNYVYWYQQLPRTAPKLLIY
RNNQRPSGVPDRFSGSKSGTSASLAISGLRSEDEADYYCAAWDDSLSAWV
FGGGTKLTVL (SEQ. ID. NO:23)

>2020Ep64-c08
V_{H}
QMQLVQSGAEVKKPGSSVKVSCKASGGTFSSYAISWVRQAPRQGLEWMGR
IIPILGIANYAQKFQGRVTITADKSTSTAYMELSSLRSEDTAVYYCASSY
GGNQLWGQGTLVTVSS (SEQ.ID.NO:24)
V_{L}
QSVLTQPPSASGTPGQRVTISCSGSDSNIGTNTVNWYQQVPGTAPKVLIY
STHQRPSGVPDRFSASKSGTSASLAISGLQSEDEADYYCSSWDVSLNAWV
FGGGTKVTVL (SEQ.ID.NO:25)

>2020Ep66-a07
V_{H}
EVQLVQSGAEVKKPGSSVKVSCKASGGTFSSYAISWVRQAPGQGLEWMGG
IIPIFGTANYAQKFQGRVTITADESTSTAYMELSSLRSEDTAVYYCASGG
DGYNYDYYYYGMDVWGQGTTVTVSS(SEQ.ID.NO:26)
V_{L}
SYELTQPPSVSVSPGQTATITCSGEKLGDKYSFWYQQKPGQSPVMVMYQD
DQRPSGTPERFSGSNSGNTATLTISGTRATDEADYYCQAWDNSASVFGSG
TKLTVL (SEQ.ID.NO:27)

>2020Ep71-e04
V_{H}
QVQLVQSGGGVVQPGRSLRLSCAASGFTFSSYGMHWVRQAPGKGLEWVAV
ISYDGSNKYYADSVKGRFTISRDNSKNTLYLQMNSLRAEDTAVYYCAKDR
AYYYGYYYYYYGMDVWGQGTTVTVSS
(SEQ.ID.NO:28)
V_{L}
QSVLTQPPSVSGAPGQRVTISCTGSSSNIGAGYDVHWYQQLPGTAPKLLI
YGNSNRPSGVPDRFSGSKSGTSASLAITGLQAEDEADYYCATWDDSLSGP
VFGGGTKVTVL (SEQ.ID.NO:29)

>2020Ep75-e02
V_{H}
QMQLVQSGAEVKKPGSSVKVSCKASGGTFSSYAISWVRQAPGQGLEWMGG
IIPIFGTANYAQKFQGRVTITADKSTSTAYMELSSLRSEDTAVYYCARPR
DYRAQYYFDYWGQGTLVTVSS (SEQ.ID.NO:30)
V_{L}
DIVMTQSPSSLSASVGDRVTITCRAGQDIDTSVNWYQLKPGKAPRLLIYA
SSSLQTGVPSRFSGGGSGAEFTLTISSLQPEDFATYFCQQTFSTSVTFGG
GTKVEIK (SEQ.ID.NO:31)

An “epitope” refers to the site on a target antigen that is recognized and bound by an antibody. The site can be entirely composed of amino acid components, entirely composed of chemical modifications of amino acids of the protein (e.g., glycosyl moieties), or composed of combinations thereof. Overlapping epitopes include at least one common amino acid residue. An epitope can be linear, which is typically 6-15 amino acids in length. Alternatively, the epitope can be conformational. The epitope to which an antibody binds can be determined by routine technology, for example, the epitope mapping method (see, e.g., descriptions below). An antibody that binds the same epitope as an exemplary antibody described herein may bind to exactly the same epitope or a substantially overlapping epitope (e.g., containing less than 3 non-overlapping amino acid residues, less than 2 non-overlapping amino acid residues, or only 1 non-overlapping amino acid residue) as the exemplary antibody. Whether two antibodies compete against each other from binding to the cognate antigen can be determined by a competition assay, which is well known in the art.

In some examples, the anti-S1 antibody comprises the same V_{H} and/or V_{L} CDRs as an exemplary antibody described herein. Two antibodies having the same V_{H} and/or V_{L} CDRs means that their CDRs are identical when determined by the same approach (e.g., the Kabat approach, the Chothia approach, the AbM approach, the Contact approach, or the IMGT approach as known in the art. See, e.g., bioinf.org.uk/abs/). Such anti-S1 antibodies may have the same V_{H}, the same V_{L}, or both as compared to an exemplary antibody described herein.

Also within the scope of the present disclosure are functional variants of any of the exemplary anti-S1 antibodies as disclosed herein. Such functional variants are substantially similar to the exemplary antibody, both structurally and functionally. A functional variant comprises substantially the same V_{H} and V_{L} CDRs as the exemplary antibody. For example, it may comprise only up to 8 (e.g., 8, 7, 6, 5, 4, 3, 2, or 1) amino acid residue variations in the total CDR regions of the antibody and binds the same protein with substantially similar affinity (e.g., having a K_{D} value in the same order). In some instances, the functional variants may have the same heavy chain CDR3 as the exemplary antibody, and optionally the same light chain CDR3 as the exemplary antibody. Alternatively or in addition, the functional variants may have the same heavy chain CDR2 as the exemplary antibody. Such an anti-S1 antibody may comprise a V_{H} fragment having CDR amino acid residue variations in only the heavy chain CDR1 as compared with the V_{H} of the exemplary antibody. In some examples, the anti-S1 antibody may further comprise a V_{L} fragment having the same V_{L} CDR3, and optionally same V_{L} CDR1 or VL CDR₂ as the exemplary antibody.

Alternatively or in addition, the amino acid residue variations can be conservative amino acid residue substitutions. As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g. Molecular Cloning: A Laboratory Manual, J. Sambrook, et al., eds., Second Edition, Cold Spring Harbor Laboratory Press, Cold Spring Harbor, New York, 1989, or Current Protocols in Molecular Biology, F.M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

In some embodiments, the anti-S1 antibody may comprise heavy chain CDRs that are at least 80% (e.g., 85%, 90%, 95%, or 98%) sequence identity, individually or collectively, as compared with the V_{H} CDRs of an exemplary antibody described herein. Alternatively or in addition, the anti-S1 antibody may comprise light chain CDRs that are at least 80% (e.g., 85%, 90%, 95%, or 98%) sequence identity, individually or collectively, as compared with the V_{L} CDRs as an exemplary antibody described herein. As used herein, “individually” means that one CDR of an antibody shares the indicated sequence identity relative to the corresponding CDR of the exemplary antibody. “Collectively” means that three V_{H} or V_{L} CDRs of an antibody in combination share the indicated sequence identity relative the corresponding three V_{H} or V_{L} CDRs of the exemplary antibody in combination.

The “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of interest. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

In some embodiments, the heavy chain of any of the anti-S1 antibodies as described herein may further comprise a heavy chain constant region (CH) or a portion thereof (e.g., CH1, CH2, CH3, or a combination thereof). The heavy chain constant region can of any suitable origin, e.g., human, mouse, rat, or rabbit. Alternatively or in addition, the light chain of the anti-S1 antibody may further comprise a light chain constant region (CL), which can be any CL known in the art. In some examples, the CL is a kappa light chain. In other examples, the CL is a lambda light chain. Antibody heavy and light chain constant regions are well known in the art, e.g., those provided in the antibody rules described at the Bioinformatics and Computational Biology group website at University College London; the vbase2 website, or the IMGT®, the international ImMunoGeneTics information system® website) both of which are incorporated by reference herein.

In some embodiments, the anti-S1 antibody disclosed herein may be a single chain antibody (scFv). A scFv antibody may comprise a V_{H} fragment and a V_{L} fragment, which may be linked via a flexible peptide linker. In some instances, the scFv antibody may be in the V_{H}➔V_{L} orientation (from N-terminus to C-terminus). In other instances, the scFv antibody may be in the V_{L}➔V_{H} orientation (from N-terminus to C-terminus). Exemplary scFv anti-S1 antibodies are provided below (V_{H}-V_{L} orientation; CDRs in boldface and peptide linker underlined):

>2020Ep53-d06
VH:
QVQLVQSGGGWQPGRSLRLSCAASGFTFSSYAMHWVRQAPGKGLEWVAVI
SYDGSNKYYADSVKGRFTISRDNSKNTLYLQMNSLRAEDTAVYYCARDGE
DGYNYISPFDYWGQGTLVTVSS
(SEQ.ID.NO:1)
VL:
SYELTQPPSVSVAPGETARITCRGNDIGSKSVHWYQQKPGQAPVLVLYYD
SDRPSGIPERFSGSNSGNTATLTISSVEAGDEADYYCQVWDIDVVFGGGT
KLTVL (SEQ.ID.NO:2)
ScFv:
QVQLVQSGGGWQPGRSLRLSCAASGFTFSSYAMHWVRQAPGKGLEWVAVI
SYDGSNKYYADSVKGRFTISRDNSKNTLYLQMNSLRAEDTAVYYCARDGE
DGYNYISPFDYWGQGTLVTVSSGGGGSGGGGSGGGGSSYELTQPPSVSVA
PGETARITCRGNDIGSKSVHWYQQKPGQAPVLVLYYDSDRPSGIPERFSG
SNSGNTATLTISSVEAGDEADYYCQVWDIDVVFGGGTKLTVL
(SEQ.ID.NO:32)

>2020Ep54-h01
VH:
QVQLVESGGGVVQPGRSLRLSCAASGFTFSSYGMHWVRQAPGKGLEWVAV
ISYDGSNKYYADSVKGRFTISRDNSKNTLYLQMNSLRAEDTAVYYCAKGR
GWYLDYWGQGTLVTVSS (SEQ.ID.NO:3)
VL:
NFMLTQPPSVSVSPGQTASITCSGDKLGDKYACWYQQKPGQSPVLVMYED
YKRPSGIPARFSGSNSGNTATLTISRVEGGDEGDYYCQVWDSRGPEVIFG
GGTKLTVL (SEQ.ID.NO:4)
ScFv:
QVQLVESGGGVVQPGRSLRLSCAASGFTFSSYGMHWVRQAPGKGLEWVAV
ISYDGSNKYYADSVKGRFTISRDNSKNTLYLQMNSLRAEDTAVYYCAKGR
GWYLDYWGQGTLVTVSSGGGGSGGGGSGGGGSNFMLTQPPSVSVSPGQTA
SITCSGDKLGDKYACWYQQKPGQSPVLVMYEDYKRPSGIPARFSGSNSGN
TATLTISRVEGGDEGDYYCQVWDSRGPEVIFGGGTKLTVL
(SEQ.ID.NO:33)

>2020Ep54-e12
VH:
EVQLVESGGGLVQPGGSLRLSCAVTGFIVSSNYMNWVRQAPGKGLEWVSV
IYSGGTTYYADSVKGRFTISRDKSKNTLYLQMNNLRAEDTAMYYCATLGN
DYGDYGTDYWGQGTLVTVSS (SEQ.ID.NO:5)
VL:
QLVLTQPPSVSVSPGQTASISCSGDRLGQKYTSWYQQRPGQSPVLIMYQD
NKRPSGIPERFSGSNSGNTATLTISGIQSMDEADYYCQVWDSGSDHAVFG
GGTQLTVL (SEQ.ID.NO:6)
ScFv:
EVQLVESGGGLVQPGGSLRLSCAVTGFIVSSNYMNWVRQAPGKGLEWVSV
IYSGGTTYYADSVKGRFTISRDKSKNTLYLQMNNLRAEDTAMYYCATLGN
DYGDYGTDYWGQGTLVTVSSGGGGSGGGGSGGGGSQLVLTQPPSVSVSPG
QTASISCSGDRLGQKYTSWYQQRPGQSPVLIMYQDNKRPSGIPERFSGSN
SGNTATLTISGIQSMDEADYYCQVWDSGSDHAVFGGGTQLTVL
(SEQ.ID.NO:34)

>2020Ep54-b12
VH:
EVQLVESGGGLVQPGGSLRLSCAASGFAFSSYAMSWVRQAPGKGLEWVSA
ISGSGGSTYYADSVKGRFTISRDNSKNTLYLQMNSLRAEDTAVYYCAKVT
RVGFDYWGQGTLVTVSS (SEQ.ID.NO:7)
VL:
SYELTQPPSVSVAPGKTARITCGGNNIGSKSVHWYQQKPGQAPVLVIYYD
SDRPSGIPERFSGSNSGNTATLTISRVEAEDEADYYCQVWDSSSDVVFGG
GTKLTVL (SEQ.ID.NO:8)
ScFv:
EVQLVESGGGLVQPGGSLRLSCAASGFAFSSYAMSWVRQAPGKGLEWVSA
ISGSGGSTYYADSVKGRFTISRDNSKNTLYLQMNSLRAEDTAVYYCAKVT
RVGFDYWGQGTLVTVSSGGGGSGGGGSGGGGSSYELTQPPSVSVAPGKTA
RITCGGNNIGSKSVHWYQQKPGQAPVLVIYYDSDRPSGIPERFSGSNSGN
TATLTISRVEAEDEADYYCQVWDSSSDVVFGGGTKLTVL
(SEQ.ID.NO:35)

>2020Ep54-b02
VH:
QVQLVQSGAEVKKPGASVKVSCKASGYTFIGYYMHWMRQAPGQGLEWMGR
INPNSGGTNYAEKFQGRVTMTRDTSISTAYMELSRLTFNDTAVYYCARWD
SSSWKFDSWGQGTLVTVSS (SEQ.ID.NO:9)
VL:
EIVLTQSPSSLSASVGDRVTITCQASQDISNYLNWYQQKPGKAPKLLIYD
ASNLETGVPSRFSGSGSGTDFTFTISSLQPEDIATYYCQQYDNLPITFGQ
GTRLEIK (SEQ.ID.NO:10)
ScFv:
QVQLVQSGAEVKKPGASVKVSCKASGYTFIGYYMHWMRQAPGQGLEWMGR
INPNSGGTNYAEKFQGRVTMTRDTSISTAYMELSRLTFNDTAVYYCARWD
SSSWKFDSWGQGTLVTVSSGGGGSGGGGSGGGGSEIVLTQSPSSLSASVG
DRVTITCQASQDISNYLNWYQQKPGKAPKLLIYDASNLETGVPSRFSGSG
SGTDFTFTISSLQPEDIATYYCQQYDNLPITFGQGTRLEIK
(SEQ.ID.NO:36)

>2020Ep54-e10
VH:
QVQLVQSGAEVRKPGSSVKVSCKASGGTFSSFAISWVRQAPGQGLEWMGG
IIPIFDTATYAQNFQGRVRMTADESTNTAYMELSSLTSEDTAVYYCARST
SYYDSRGDYKVGDFDYWGQGTLVTVSS(SEQ.ID.NO:11)
VL:
AIRMTQSPSSLSASVGDRVTITCQASQDISNYLNWYQQKPGKAPKLLIYD
ASNLETGVPSRFSGSGSGTDFTFTISSLQPEDIATYYCQQYDNLPLTFGG
GTKVEIK (SEQ.ID.NO:12)
ScFv:
QVQLVQSGAEVRKPGSSVKVSCKASGGTFSSFAISWVRQAPGQGLEWMGG
IIPIFDTATYAQNFQGRVRMTADESTNTAYMELSSLTSEDTAVYYCARST
SYYDSRGDYKVGDFDYWGQGTLVTVSSGGGGSGGGGSGGGGSAIRMTQSP
SSLSASVGDRVTITCQASQDISNYLNWYQQKPGKAPKLLIYDASNLETGV
PSRFSGSGSGTDFTFTISSLQPEDIATYYCQQYDNLPLTFGGGTKVEIK
(SEQ.ID.NO:37)

>2020Ep60-f05
VH:
QVQLVESGGGVVQPGGSLRLSCAASGFTFSSYGMHWVRQAPGKGLEWVAV
ISYDGSNKYYADSVKGRFTISRDNSKNTLYLQMNSLRAEDTAVYYCAKAR
VALGYFDWLLYDGMDVWGQGTTVTVSS(SEQ.ID.NO:13)
VL:
SSELTQPPSVSVSPGQTASITCSGDKLGNKYVFWYQQKPGQSPILVIYQD
NRRPSGIPERFSGSNSGNTATLTISGDPAMDEADYYCQAWDSSTVVFGGG
TKLTVL (SEQ.ID.NO:14)
ScFv:
QVQLVESGGGVVQPGGSLRLSCAASGFTFSSYGMHWVRQAPGKGLEWVAV
ISYDGSNKYYADSVKGRFTISRDNSKNTLYLQMNSLRAEDTAVYYCAKAR
VALGYFDWLLYDGMDVWGQGTTVTVSSGGGGSGGGGSGGGGSSSELTQPP
SVSVSPGQTASITCSGDKLGNKYVFWYQQKPGQSPILVIYQDNRRPSGIP
ERFSGSNSGNTATLTISGDPAMDEADYYCQAWDSSTVVFGGGTKLTVL
(SEQ.ID.NO:38)

>2020Ep60-a12
VH:
EVQLVESGGGVVQPGRSLRLSCAASGFTFSSYGMHWVRQAPGKGLEWVAV
ISYDGSNKYYADSVKGRFTISRDNSKNTLYLQMNSLRAEDTAVYYCAKGL
EYYFDYWGQGTLVTVSS (SEQ.ID.NO:15)
VL:
SYELTQPPSLSVSPGQTARITCSGDALPEQYAYWYQQKPGQAPVLVIYKD
SERPSGIPERFSGSSSGTTVTLTISGVQAEDEADYYCQAWDSSTVVFGGG
TKLTVL (SEQ.ID.NO:16)
ScFv:
EVQLVESGGGVVQPGRSLRLSCAASGFTFSSYGMHWVRQAPGKGLEWVAV
ISYDGSNKYYADSVKGRFTISRDNSKNTLYLQMNSLRAEDTAVYYCAKGL
EYYFDYWGQGTLVTVSSGGGGSGGGGSGGGGSSYELTQPPSLSVSPGQTA
RITCSGDALPEQYAYWYQQKPGQAPVLVIYKDSERPSGIPERFSGSSSGT
TVTLTISGVQAEDEADYYCQAWDSSTVVFGGGTKLTVL
(SEQ.ID.NO:39)

>2020Ep61-a08
VH:
QVQLVQSGAEVKKPGASVKVSCKASGYTFTSYYMHWVRQAPGQGLEWMGI
INPSGGSTSYAQKFQGRVTMTRDTSTSTVYMELSSLRSEDTAVYYCARES
GNYYYDSSGYTFDYWGQGTLVTVSS(SEQ.ID.NO:17)
VL:
SYVLTQPLSVSVALGQTARITCGGNNIGSKNVHWYQQKPGQAPVLVIYYD
SDRPSGIPERFSGSNSGNTATLTISGTQAMDEADYYCQAWDSSTVVFGGG
TKLTVL (SEQ.ID.NO:18)
ScFv:
QVQLVQSGAEVKKPGASVKVSCKASGYTFTSYYMHWVRQAPGQGLEWMGI
INPSGGSTSYAQKFQGRVTMTRDTSTSTVYMELSSLRSEDTAVYYCARES
GNYYYDSSGYTFDYWGQGTLVTVSSGGGGSGGGGSGGGGSSYVLTQPLSV
SVALGQTARITCGGNNIGSKNVHWYQQKPGQAPVLVIYYDSDRPSGIPER
FSGSNSGNTATLTISGTQAMDEADYYCQAWDSSTVVFGGGTKLTVL
(SEQ.ID.NO:40)

>2020Ep61-c12
VH:
EVQLVESGGGLVQPGGSLRLSCAASGFTFSSYAMSWVRQAPGKGLEWVSA
ISGSGGSTYYADSVKGRFTISRDNSKNTLYLQMNSLRAEDTAVYYCAKDE
YSYGSLYFDYWGQGTLVTVSS (SEQ.ID.NO:19)
VL:
SYELTQPPSVSVAPGQTARITCGGNNIGSKSVHWYQQKPGQAPVLWYDDS
DRPSGIPERFSGSNSGNTATLTISRVEAGDEADYYCQVWDSSSDSVVFGG
GTKLTVL (SEQ.ID.NO:20)
ScFv:
EVQLVESGGGLVQPGGSLRLSCAASGFTFSSYAMSWVRQAPGKGLEWVSA
ISGSGGSTYYADSVKGRFTISRDNSKNTLYLQMNSLRAEDTAVYYCAKDE
YSYGSLYFDYWGQGTLVTVSSGGGGSGGGGSGGGGSSYELTQPPSVSVAP
GQTARITCGGNNIGSKSVHWYQQKPGQAPVLVVYDDSDRPSGIPERFSGS
NSGNTATLTISRVEAGDEADYYCQVWDSSSDSVVFGGGTKLTVL
(SEQ.ID.NO:41)

>2020Ep64-g10
VH:
QVQLVQSGGGWQPGRSLRLSCAASGFTFSSYAMHWVRQAPGKGLEWVAVI
SYDGSNKYYADSVKGRFTISRDNSKNTLYLQMNSLRAEDTAVYYCARDGE
DGYNYISPFDYWGQGTLVTVSS(SEQ.ID.NO:1)
VL:
SYELTQPPSVSVAPGETARITCRGNDIGSKSVHWYQQKPGQAPVLVLYYD
SDRPSGIPERFSGSNSGNTATLTISSVEAGDEADYYCHVWDIDVVFGGGT
KLTVL (SEQ.ID.NO:21)
ScFv:
QVQLVQSGGGWQPGRSLRLSCAASGFTFSSYAMHWVRQAPGKGLEWVAVI
SYDGSNKYYADSVKGRFTISRDNSKNTLYLQMNSLRAEDTAVYYCARDGE
DGYNYISPFDYWGQGTLVTVSSGGGGSGGGGSGGGRSSYELTQPPSVSVA
PGETARITCRGNDIGSKSVHWYQQKPGQAPVLVLYYDSDRPSGIPERFSG
SNSGNTATLTISSVEAGDEADYYCHVWDIDVVFGGGTKLTVL
(SEQ.ID.NO:42)

>2020Ep66-d03
VH:
QVQLQQWGAGLLKPSETLSLTCAVYGGSFSGYYWSWIRQPPGKGLEWIGE
INHSGSTNYNPSLKSRVTISVDTSKNQFSLKLSSVTAADTAVYYCARGPP
LWNYGKGFDYWGQGTLVTVSS (SEQ.ID.NO:22)
VL:
QPVLTQPPSASGTPGQRVTISCSGSSSNIGSNYVYWYQQLPRTAPKLLIY
RNNQRPSGVPDRFSGSKSGTSASLAISGLRSEDEADYYCAAWDDSLSAWV
FGGGTKLTVL (SEQ.ID.NO:23)
ScFv:
QVQLQQWGAGLLKPSETLSLTCAVYGGSFSGYYWSWIRQPPGKGLEWIGE
INHSGSTNYNPSLKSRVTISVDTSKNQFSLKLSSVTAADTAVYYCARGPP
LWNYGKGFDYWGQGTLVTVSSGGGGSGKGGSGGGGSQPVLTQPPSASGTP
GQRVTISCSGSSSNIGSNYVYWYQQLPRTAPKLLIYRNNQRPSGVPDRFS
GSKSGTSASLAISGLRSEDEADYYCAAWDDSLSAWVFGGGTKLTVL
(SEQ.ID.NO:43)

>2020Ep64-c08
VH:
QMQLVQSGAEVKKPGSSVKVSCKASGGTFSSYAISWVRQAPRQGLEWMGR
IIPILGIANYAQKFQGRVTITADKSTSTAYMELSSLRSEDTAVYYCASSY
GGNQLWGQGTLVTVSS (SEQ.ID.NO:24)
VL:
QSVLTQPPSASGTPGQRVTISCSGSDSNIGTNTVNWYQQVPGTAPKVLIY
STHQRPSGVPDRFSASKSGTSASLAISGLQSEDEADYYCSSWDVSLNAWV
FGGGTKVTVL (SEQ.ID.NO:25)
ScFv:
QMQLVQSGAEVKKPGSSVKVSCKASGGTFSSYAISWVRQAPRQGLEWMGR
IIPILGIANYAQKFQGRVTITADKSTSTAYMELSSLRSEDTAVYYCASSY
GGNQLWGQGTLVTVSSGGGGSGGGGSGGGGSQSVLTQPPSASGTPGQRVT
ISCSGSDSNIGTNTVNWYQQVPGTAPKVLIYSTHQRPSGVPDRFSASKSG
TSASLAISGLQSEDEADYYCSSWDVSLNAWVFGGGTKVTVL
(SEQ.ID.NO:44)

>2020Ep66-a07
VH:
EVQLVQSGAEVKKPGSSVKVSCKASGGTFSSYAISWVRQAPGQGLEWMGG
IIPIFGTANYAQKFQGRVTITADESTSTAYMELSSLRSEDTAVYYCASGG
DGYNYDYYYYGMDVWGQGTTVTVSS
(SEQ.ID.NO:26)
VL:
SYELTQPPSVSVSPGQTATITCSGEKLGDKYSFWYQQKPGQSPVMVMYQD
DQRPSGTPERFSGSNSGNTATLTISGTRATDEADYYCQAWDNSASVFGSG
TKLTVL (SEQ.ID.NO:27)
ScFv:
EVQLVQSGAEVKKPGSSVKVSCKASGGTFSSYAISWVRQAPGQGLEWMGG
IIPIFGTANYAQKFQGRVTITADESTSTAYMELSSLRSEDTAVYYCASGG
DGYNYDYYYYGMDVNGQGTTVTVSSGGGGSGGGGSGGGGSSYELTQPPSV
SVSPGQTATITCSGEKLGDKYSFWYQQKPGQSPVMVMYQDDQRPSGTPER
FSGSNSGNTATLTISGTRATDEADYYCQAWDNSASVFGSGTKLTVL
(SEQ.ID.NO:45)

>2020Ep71-e04
VH:
QVQLVQSGGGVVQPGRSLRLSCAASGFTFSSYGMHWVRQAPGKGLEWVAV
ISYDGSNKYYADSVKGRFTISRDNSKNTLYLQMNSLRAEDTAVYYCAKDR
AYYYGYYYYYYGMDVWGQGTTVTVSS (SEQ.ID.NO:28)
VL:
QSVLTQPPSVSGAPGQRVTISCTGSSSNIGAGYDVHWYQQLPGTAPKLLI
YGNSNRPSGVPDRFSGSKSGTSASLAITGLQAEDEADYYCATWDDSLSGP
VFGGGTKVTVL (SEQ.ID.NO:29)
ScFv:
QVQLVQSGGGVVQPGRSLRLSCAASGFTFSSYGMHWVRQAPGKGLEWVAV
ISYDGSNKYYADSVKGRFTISRDNSKNTLYLQMNSLRAEDTAVYYCAKDR
AYYYGYYYYYYGMDVWGQGTTVTVSSGGGGSGGGGSGGGGSQSVLTQPPS
VSGAPGQRVTISCTGSSSNIGAGYDVHWYQQLPGTAPKLLIYGNSNRPSG
VPDRFSGSKSGTSASLAITGLQAEDEADYYCATWDDSLSGPVFGGGTKVT
VL (SEQ.ID.NO:46)

>2020Ep75-e02
VH:
QMQLVQSGAEVKKPGSSVKVSCKASGGTFSSYAISWVRQAPGQGLEWMGG
IIPIFGTANYAQKFQGRVTITADKSTSTAYMELSSLRSEDTAVYYCARPR
DYRAQYYFDYWGQGTLVTVSS (SEQ.ID.NO:30)
VL:
DIVMTQSPSSLSASVGDRVTITCRAGQDIDTSVNWYQLKPGKAPRLLIYA
SSSLQTGVPSRFSGGGSGAEFTLTISSLQPEDFATYFCQQTFSTSVTFGG
GTKVEIK (SEQ.ID.NO:31)
ScFv:
QMQLVQSGAEVKKPGSSVKVSCKASGGTFSSYAISWVRQAPGQGLEWMGG
IIPIFGTANYAQKFQGRVTITADKSTSTAYMELSSLRSEDTAVYYCARPR
DYRAQYYFDYWGQGTLVTVSSGGGGSGGGGSGGGGSDIVMTQSPSSLSAS
VGDRVTITCRAGQDIDTSVNWYQLKPGKAPRLLIYASSSLQTGVPSRFSG
GGSGAEFTLTISSLQPEDFATYFCQQTFSTSVTFGGGTKVEIK
(SEQ.ID.NO:47)

Any of the anti-S1 antibodies as described herein, e.g., the exemplary anti-S1 antibodies provided here, can bind and inhibit (e.g., reduce or eliminate) the ability of SARS-CoV-2 to bind to or infect cells, particularly cells expressing ACE2. In some embodiments, the anti-S1 antibody as described herein can bind and inhibit ability of SARS-CoV-2 to infect cells by at least 30% (e.g., 35%, 40%, 45%, 50%, 60%, 70%, 80%, 90%, 95% or greater, including any increment therein). The inhibitory activity of an anti-S1 antibody described herein can be determined by routine methods known in the art, e.g., by an assay for measuring the Kᵢ,^{app} value.

In some examples, the Kᵢ,^{app} value of an antibody may be determined by measuring the inhibitory effect of different concentrations of the antibody on the extent of a relevant reaction; fitting the change in pseudo-first order rate constant (v) as a function of inhibitor concentration to the modified Morrison equation (Equation 1) yields an estimate of the apparent Ki value. For a competitive inhibitor, the Kᵢ^{app} can be obtained from the y-intercept extracted from a linear regression analysis of a plot of Kᵢ,^{app} versus substrate concentration.

$$v = A ⋅ \frac{\left(\left[E\right] - \left[I\right] - {K}_{i}^{a p p}\right) + \sqrt{{\left(\left[E\right] - \left[I\right] - {K}_{i}^{a p p}\right)}^{2} + 4 \left[E\right] ⋅ {K}_{i}^{a p p}}}{2}$$

Where A is equivalent to νₒ/E, the initial velocity (νₒ) of the enzymatic reaction in the absence of inhibitor (I) divided by the total enzyme concentration (E). In some embodiments, the anti-S1 antibody described herein may have a Ki^{app} value of 1000, 500, 100, 50, 40, 30, 20, 10, 5 pM or less for the target antigen or antigen epitope.

### II. Preparation of Anti-S1 Antibodies

Antibodies capable of binding S1 or the RBD as described herein can be made by any method known in the art. See, for example, Harlow and Lane, (1998) Antibodies: A Laboratory Manual, Cold Spring Harbor Laboratory, New York. In some embodiments, the antibody may be produced by the conventional hybridoma technology. Alternatively, the anti-S1 antibody may be identified from a suitable library (e.g., a human antibody library).

In some instances, high affinity fully human S1 or RBD binders may be obtained from a human antibody library following the screening strategy illustrated in Example 1. This strategy allows for maximizing the library diversity to cover S1 or the RBD.

If desired, an antibody (monoclonal or polyclonal) of interest (e.g., produced by a hybridoma cell line or isolated from an antibody library) may be sequenced and the polynucleotide sequence may then be cloned into a vector for expression or propagation. The sequence encoding the antibody of interest may be maintained in vector in a host cell and the host cell can then be expanded and frozen for future use. In an alternative, the polynucleotide sequence may be used for genetic manipulation to, e.g., humanize the antibody or to improve the affinity (affinity maturation), or other characteristics of the antibody. For example, the constant region may be engineered to more resemble human constant regions to avoid immune response if the antibody is from a non-human source and is to be used in clinical trials and treatments in humans. Alternatively or in addition, it may be desirable to genetically manipulate the antibody sequence to obtain greater affinity and/or specificity to the target antigen and greater efficacy in blocking the binding of S1 to ACE2. It will be apparent to one of skill in the art that one or more polynucleotide changes can be made to the antibody and still maintain its binding specificity to the target antigen.

Alternatively, antibodies capable of binding to the target antigens as described herein (a S1 or RBD molecule) may be isolated from a suitable antibody library via routine practice. Antibody libraries can be used to identify proteins that bind to a target antigen (RBD and/or S1) via routine screening processes. In the selection process, the polypeptide component is probed with the target antigen or a fragment thereof and, if the polypeptide component binds to the target, the antibody library member is identified, typically by retention on a support. Retained display library members are recovered from the support and analyzed. The analysis can include amplification and a subsequent selection under similar or dissimilar conditions. For example, positive and negative selections can be alternated. The analysis can also include determining the amino acid sequence of the polypeptide component and purification of the polypeptide component for detailed characterization.

There are a number of routine methods known in the art to identify and isolate antibodies capable of binding to the target antigens described herein, including phage display, yeast display, ribosomal display, or mammalian display technology.

Antigen-binding fragments of an intact antibody (full-length antibody) can be prepared via routine methods. For example, F(ab′)2 fragments can be produced by pepsin digestion of an antibody molecule, and Fab fragments that can be generated by reducing the disulfide bridges of F(ab′)2 fragments.

Genetically engineered antibodies, such as humanized antibodies, chimeric antibodies, single-chain antibodies, and bi-specific antibodies, can be produced via, e.g., conventional recombinant technology. In one example, DNA encoding a monoclonal antibodies specific to a target antigen can be readily isolated and sequenced using conventional procedures (e.g., by using oligonucleotide probes that are capable of binding specifically to genes encoding the heavy and light chains of the monoclonal antibodies). Once isolated, the DNA may be placed into one or more expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. See, e.g., PCT Publication No. WO 87/04462. The DNA can then be modified, for example, by substituting the coding sequence for human heavy and light chain constant domains in place of the homologous murine sequences, Morrison et al., (1984) Proc. Nat. Acad. Sci. 81:6851, or by covalently joining to the immunoglobulin coding sequence all or part of the coding sequence for a non-immunoglobulin polypeptide. In that manner, genetically engineered antibodies, such as “chimeric” or “hybrid” antibodies; can be prepared that have the binding specificity of a target antigen.

Techniques developed for the production of “chimeric antibodies” are well known in the art. See, e.g., Morrison et al. (1984) Proc. Natl. Acad. Sci. USA 81, 6851; Neuberger et al. (1984) Nature 312, 604; and Takeda et al. (1984) Nature 314:452.

Methods for constructing humanized antibodies are also well known in the art. See, e.g., Queen et al., Proc. Natl. Acad. Sci. USA, 86:10029-10033 (1989). In one example, variable regions of V_{H} and V_{L} of a parent non-human antibody are subjected to three-dimensional molecular modeling analysis following methods known in the art. Next, framework amino acid residues predicted to be important for the formation of the correct CDR structures are identified using the same molecular modeling analysis. In parallel, human V_{H} and V_{L} chains having amino acid sequences that are homologous to those of the parent non-human antibody are identified from any antibody gene database using the parent V_{H} and V_{L} sequences as search queries. Human V_{H} and V_{L} acceptor genes are then selected.

The CDR regions within the selected human acceptor genes can be replaced with the CDR regions from the parent non-human antibody or functional variants thereof. When necessary, residues within the framework regions of the parent chain that are predicted to be important in interacting with the CDR regions (see above description) can be used to substitute for the corresponding residues in the human acceptor genes.

A single-chain antibody can be prepared via recombinant technology by linking a nucleotide sequence coding for a heavy chain variable region and a nucleotide sequence coding for a light chain variable region. Preferably, a flexible linker is incorporated between the two variable regions. Alternatively, techniques described for the production of single chain antibodies (U.S. Pat. Nos. 4,946,778 and 4,704,692) can be adapted to produce a phage-display, yeast-display, mammalian cell-display, or mRNA-display scFv library and scFv clones specific to S1 can be identified from the library following routine procedures. Positive clones can be subjected to further screening to identify those that block the ability of S1 to bind ACE2.

Antibodies obtained following a method known in the art and described herein can be characterized using methods well known in the art. For example, one method is to identify the epitope to which the antigen binds, or “epitope mapping.” There are many methods known in the art for mapping and characterizing the location of epitopes on proteins, including solving the crystal structure of an antibody-antigen complex, competition assays, gene fragment expression assays, and synthetic peptide-based assays, as described, for example, in Chapter 11 of Harlow and Lane, Using Antibodies, a Laboratory Manual, Cold Spring Harbor Laboratory Press, Cold Spring Harbor, N.Y., 1999. In an additional example, epitope mapping can be used to determine the sequence, to which an antibody binds. The epitope can be a linear epitope, i.e., contained in a single stretch of amino acids, or a conformational epitope formed by a three-dimensional interaction of amino acids that may not necessarily be contained in a single stretch (primary structure linear sequence). Peptides of varying lengths (e.g., at least 4-6 amino acids long) can be isolated or synthesized (e.g., recombinantly) and used for binding assays with an antibody. In another example, the epitope to which the antibody binds can be determined in a systematic screening by using overlapping peptides derived from the target antigen sequence and determining binding by the antibody. According to the gene fragment expression assays, the open reading frame encoding the target antigen is fragmented either randomly or by specific genetic constructions and the reactivity of the expressed fragments of the antigen with the antibody to be tested is determined. The gene fragments may, for example, be produced by PCR and then transcribed and translated into protein in vitro, in the presence of radioactive amino acids. The binding of the antibody to the radioactively labeled antigen fragments is then determined by immunoprecipitation and gel electrophoresis. Certain epitopes can also be identified by using large libraries of random peptide sequences displayed on the surface of phage particles (phage libraries).

Alternatively, a defined library of overlapping peptide fragments can be tested for binding to the test antibody in simple binding assays. In an additional example, mutagenesis of an antigen binding domain, domain swapping experiments and alanine scanning mutagenesis can be performed to identify residues required, sufficient, and/or necessary for epitope binding. For example, domain swapping experiments can be performed using a mutant of a target antigen in which various fragments of S1 have been replaced (swapped) with sequences from a closely related, but antigenically distinct protein. By assessing binding of the antibody to the mutant S1, the importance of the particular antigen fragment to antibody binding can be assessed.

Alternatively, competition assays can be performed using other antibodies known to bind to the same antigen to determine whether an antibody binds to the same epitope as the other antibodies. Competition assays are well known to those of skill in the art.

In some examples, an anti-S1 antibody is prepared by recombinant technology as exemplified below.

Nucleic acids encoding the heavy and light chain of an anti-S1 antibody as described herein can be cloned into one expression vector, each nucleotide sequence being in operable linkage to a suitable promoter. In one example, each of the nucleotide sequences encoding the heavy chain and light chain is in operable linkage to a distinct prompter. Alternatively, the nucleotide sequences encoding the heavy chain and the light chain can be in operable linkage with a single promoter, such that both heavy and light chains are expressed from the same promoter. When necessary, an internal ribosomal entry site (IRES) can be inserted between the heavy chain and light chain encoding sequences.

In some examples, the nucleotide sequences encoding the two chains of the antibody are cloned into two vectors, which can be introduced into the same or different cells. When the two chains are expressed in different cells, each of them can be isolated from the host cells expressing such and the isolated heavy chains and light chains can be mixed and incubated under suitable conditions allowing for the formation of the antibody.

Generally, a nucleic acid sequence encoding one or all chains of an antibody can be cloned into a suitable expression vector in operable linkage with a suitable promoter using methods known in the art. For example, the nucleotide sequence and vector can be contacted, under suitable conditions, with a restriction enzyme to create complementary ends on each molecule that can pair with each other and be joined together with a ligase. Alternatively, synthetic nucleic acid linkers can be ligated to the termini of a gene. These synthetic linkers contain nucleic acid sequences that correspond to a particular restriction site in the vector. The selection of expression vectors/promoter would depend on the type of host cells for use in producing the antibodies.

A variety of promoters can be used for expression of the antibodies described herein, including, but not limited to, cytomegalovirus (CMV) intermediate early promoter, a viral LTR such as the Rous sarcoma virus LTR, HIV-LTR, HTLV-1 LTR, the simian virus 40 (SV40) early promoter, E. coli lac UV5 promoter, and the herpes simplex tk virus promoter.

Regulatable promoters can also be used. Such regulatable promoters include those using the lac repressor from E. coli as a transcription modulator to regulate transcription from lac operator-bearing mammalian cell promoters [Brown, M. et al., Cell, 49:603-612 (1987)], those using the tetracycline repressor (tetR) [Gossen, M., and Bujard, H., Proc. Natl. Acad. Sci. USA 89:5547-5551 (1992); Yao, F. et al., Human Gene Therapy, 9:1939-1950 (1998); Shockelt, P., et al., Proc. Natl. Acad. Sci. USA, 92:6522-6526 (1995)]. Other systems include FK506 dimer, VP16 or p65 using astradiol, RU486, diphenol murislerone, or rapamycin. Inducible systems are available from Invitrogen, Clontech and Ariad.

Regulatable promoters that include a repressor with the operon can be used. In one embodiment, the lac repressor from E. coli can function as a transcriptional modulator to regulate transcription from lac operator-bearing mammalian cell promoters [M. Brown et al., Cell, 49:603-612 (1987); Gossen and Bujard (1992); M. Gossen et al., Natl. Acad. Sci. USA, 89:5547-5551 (1992)] combined the tetracycline repressor (tetR) with the transcription activator (VP 16) to create a tetR-mammalian cell transcription activator fusion protein, tTa (tetR-VP 16), with the tetO-bearing minimal promoter derived from the human cytomegalovirus (hCMV) major immediate-early promoter to create a tetR-tet operator system to control gene expression in mammalian cells. In one embodiment, a tetracycline inducible switch is used. The tetracycline repressor (tetR) alone, rather than the tetR-mammalian cell transcription factor fusion derivatives can function as potent trans-modulator to regulate gene expression in mammalian cells when the tetracycline operator is properly positioned downstream for the TATA element of the CMVIE promoter (Yao et al., Human Gene Therapy, 10(16):1392-1399 (2003)). One particular advantage of this tetracycline inducible switch is that it does not require the use of a tetracycline repressor-mammalian cells transactivator or repressor fusion protein, which in some instances can be toxic to cells (Gossen et al., Natl. Acad. Sci. USA, 89:5547-5551 (1992); Shockett et al., Proc. Natl. Acad. Sci. USA, 92:6522-6526 (1995)), to achieve its regulatable effects.

Additionally, the vector can contain, for example, some or all of the following: a selectable marker gene, such as the neomycin gene for selection of stable or transient transfectants in mammalian cells; enhancer/promoter sequences from the immediate early gene of human CMV for high levels of transcription; transcription termination and RNA processing signals from SV40 for mRNA stability; SV40 polyoma origins of replication and ColE1 for proper episomal replication; internal ribosome binding sites (IRESes), versatile multiple cloning sites; and T7 and SP6 RNA promoters for in vitro transcription of sense and antisense RNA. Suitable vectors and methods for producing vectors containing transgenes are well known and available in the art.

Examples of polyadenylation signals useful to practice the methods described herein include, but are not limited to, human collagen I polyadenylation signal, human collagen II polyadenylation signal, and SV40 polyadenylation signal.

One or more vectors (e.g., expression vectors) comprising nucleic acids encoding any of the antibodies may be introduced into suitable host cells for producing the antibodies. The host cells can be cultured under suitable conditions for expression of the antibody or any polypeptide chain thereof. Such antibodies or polypeptide chains thereof can be recovered by the cultured cells (e.g., from the cells or the culture supernatant) via a conventional method, e.g., affinity purification. If necessary, polypeptide chains of the antibody can be incubated under suitable conditions for a suitable period of time allowing for production of the antibody.

In some embodiments, methods for preparing an antibody described herein involve a recombinant expression vector that encodes both the heavy chain and the light chain of an anti-S1 antibody, as also described herein. The recombinant expression vector can be introduced into a suitable host cell (e.g., a dhfr- CHO cell) by a conventional method, e.g., calcium phosphate-mediated transfection. Positive transformant host cells can be selected and cultured under suitable conditions allowing for the expression of the two polypeptide chains that form the antibody, which can be recovered from the cells or from the culture medium. When necessary, the two chains recovered from the host cells can be incubated under suitable conditions allowing for the formation of the antibody.

In one example, two recombinant expression vectors are provided, one encoding the heavy chain of the anti-S1 antibody and the other encoding the light chain of the anti-S1 antibody. Both of the two recombinant expression vectors can be introduced into a suitable host cell (e.g., dhfr- CHO cell) by a conventional method, e.g., calcium phosphate-mediated transfection. Alternatively, each of the expression vectors can be introduced into a suitable host cells. Positive transformants can be selected and cultured under suitable conditions allowing for the expression of the polypeptide chains of the antibody. When the two expression vectors are introduced into the same host cells, the antibody produced therein can be recovered from the host cells or from the culture medium. If necessary, the polypeptide chains can be recovered from the host cells or from the culture medium and then incubated under suitable conditions allowing for formation of the antibody. When the two expression vectors are introduced into different host cells, each of them can be recovered from the corresponding host cells or from the corresponding culture media. The two polypeptide chains can then be incubated under suitable conditions for formation of the antibody.

Standard molecular biology techniques are used to prepare the recombinant expression vector, transfect the host cells, select for transformants, culture the host cells and recovery of the antibodies from the culture medium. For example, some antibodies can be isolated by affinity chromatography with a Protein A or Protein G coupled matrix.

Any of the nucleic acids encoding the heavy chain, the light chain, or both of an anti-S1 antibody as described herein, vectors (e.g., expression vectors) containing such; and host cells comprising the vectors are within the scope of the present disclosure.

### III.Applications of Anti-S1 Antibodies

Any of the anti-S1 antibodies disclosed herein can be used for therapeutic, diagnostic, and/or research purposes, all of which are within the scope of the present disclosure.

### Pharmaceutical Compositions

The antibodies, as well as the encoding nucleic acids or nucleic acid sets, vectors comprising such, or host cells comprising the vectors, as described herein can be mixed with a pharmaceutically acceptable carrier (excipient) to form a pharmaceutical composition for use in treating a target disease. “Acceptable” means that the carrier must be compatible with the active ingredient of the composition (and preferably, capable of stabilizing the active ingredient) and not deleterious to the subject to be treated. Pharmaceutically acceptable excipients (carriers) including buffers, which are well known in the art. See, e.g., Remington: The Science and Practice of Pharmacy 20th Ed. (2000) Lippincott Williams and Wilkins, Ed. K. E. Hoover.

The pharmaceutical compositions to be used in the present methods can comprise pharmaceutically acceptable carriers, excipients, or stabilizers in the form of lyophilized formulations or aqueous solutions. (Remington: The Science and Practice of Pharmacy 20th Ed. (2000) Lippincott Williams and Wilkins, Ed. K. E. Hoover). Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

In some examples, the pharmaceutical composition described herein comprises liposomes containing the antibodies (or the encoding nucleic acids) which can be prepared by methods known in the art, such as described in Epstein, et al., Proc. Natl. Acad. Sci. USA 82:3688 (1985); Hwang, et al., Proc. Natl. Acad. Sci. USA 77:4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

The antibodies, or the encoding nucleic acid(s), may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are known in the art, see, e.g., Remington, The Science and Practice of Pharmacy 20th Ed. Mack Publishing (2000).

In other examples, the pharmaceutical composition described herein can be formulated in sustained-release format. Suitable examples of sustained-release preparations include semipermeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinyl alcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(-)-3-hydroxybutyric acid.

The pharmaceutical compositions to be used for in vivo administration must be sterile. This is readily accomplished by, for example, filtration through sterile filtration membranes. Therapeutic antibody compositions are generally placed into a container having a sterile access port, for example, an intravenous solution bag or vial having a stopper pierceable by a hypodermic injection needle.

The pharmaceutical compositions described herein can be in unit dosage forms such as tablets, pills, capsules, powders, granules, solutions or suspensions, or suppositories, for oral, parenteral or rectal administration, or administration by inhalation or insufflation.

For preparing solid compositions such as tablets, the principal active ingredient can be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the present invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

Suitable surface-active agents include, in particular, non-ionic agents, such as polyoxyethylenesorbitans (e.g., Tween™ 20, 40, 60, 80 or 85) and other sorbitans (e.g., Span™ 20, 40, 60, 80 or 85). Compositions with a surface-active agent will conveniently comprise between 0.05 and 5% surface-active agent, and can be between 0.1 and 2.5%. It will be appreciated that other ingredients may be added, for example mannitol or other pharmaceutically acceptable vehicles, if necessary.

Suitable emulsions may be prepared using commercially available fat emulsions, such as Intralipid™, Liposyn™, Infonutrol™, Lipofundin™ and Lipiphysan™. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g. egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion can comprise fat droplets between 0.1 and 1.0 µm, particularly 0.1 and 0.5 µm, and have a pH in the range of 5.5 to 8.0.

The emulsion compositions can be those prepared by mixing an antibody with Intralipid™ or the components thereof (soybean oil, egg phospholipids, glycerol and water).

Pharmaceutical compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect.

Compositions in preferably sterile pharmaceutically acceptable solvents may be nebulized by use of gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices which deliver the formulation in an appropriate manner.

### Therapeutic Applications

To practice the method disclosed herein, an effective amount of the pharmaceutical composition described herein can be administered to a subject (e.g., a human) in need of the treatment via a suitable route, such as intravenous administration, e.g., as a bolus or by continuous infusion over a period of time, by intramuscular, intraperitoneal, intracerebrospinal, subcutaneous, intra-articular, intrasynovial, intrathecal, oral, inhalation or topical routes. Commercially available nebulizers for liquid formulations, including jet nebulizers and ultrasonic nebulizers are useful for administration. Liquid formulations can be directly nebulized and lyophilized powder can be nebulized after reconstitution. Alternatively, the antibodies as described herein can be aerosolized using a fluorocarbon formulation and a metered dose inhaler, or inhaled as a lyophilized and milled powder.

The subject to be treated by the methods described herein can be a mammal, more preferably a human. Mammals include, but are not limited to, farm animals, sport animals, pets, primates, horses, dogs, cats, mice and rats. The subject may have, be at risk for, or be suspected of having, a target disease/disorder characterized by a coronavirus infection. The coronavirus may be SARS-CoV-2, severe acute respiratory syndrome coronavirus (SARS-CoV), or Middle East respiratory syndrome coronavirus (MERS-CoV). The coronavirus may also be human coronavirus 229E, NL63, OC43, or HKU1. In one example, the coronavirus is SARS-CoV-2. The target disease/disorder may be SARS, MERS, or COVID-19. In one example, the target disease/disorder is COVID-19.

A subject having a coronavirus infection or suspected of having the infection can be identified by routine medical examination, e.g., laboratory tests, organ functional tests, or CT scans. In one example, the subject has a SARS-CoV-2 infection or is suspected of having such an infection.

A subject suspected of having any of such target disease/disorder might show one or more symptoms of the disease/disorder. A subject at risk for the disease/disorder can be a subject having one or more of the risk factors for that disease/disorder.

As used herein, “an effective amount” refers to the amount of each active agent required to confer therapeutic effect on the subject, either alone or in combination with one or more other active agents. Determination of whether an amount of the antibody achieved the therapeutic effect would be evident to one of skill in the art. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment.

Empirical considerations, such as the half-life, generally will contribute to the determination of the dosage. For example, antibodies that are compatible with the human immune system, such as humanized antibodies or fully human antibodies, may be used to prolong half-life of the antibody and to prevent the antibody being attacked by the host’s immune system. Frequency of administration may be determined and adjusted over the course of therapy, and is generally, but not necessarily, based on treatment and/or suppression and/or amelioration and/or delay of a target disease/disorder. Alternatively, sustained continuous release formulations of an antibody may be appropriate. Various formulations and devices for achieving sustained release are known in the art.

In one example, dosages for an antibody as described herein may be determined empirically in individuals who have been given one or more administration(s) of the antibody. Individuals are given incremental dosages of the agonist. To assess efficacy of the agonist, an indicator of the disease/disorder can be followed.

Generally, for administration of any of the antibodies described herein, an initial candidate dosage can be about 2 mg/kg. For the purpose of the present disclosure, a typical daily dosage might range from about any of 0.1 µg/kg to 3 µg/kg to 30 µg/kg to 300 µg/kg to 3 mg/kg, to 30 mg/kg to 100 mg/kg or more, depending on the factors mentioned above. For repeated administrations over several days or longer, depending on the condition, the treatment is sustained until a desired suppression of symptoms occurs or until sufficient therapeutic levels are achieved to alleviate a target disease or disorder, or a symptom thereof. An exemplary dosing regimen comprises administering an initial dose of about 2 mg/kg, followed by a weekly maintenance dose of about 1 mg/kg of the antibody, or followed by a maintenance dose of about 1 mg/kg every other week. However, other dosage regimens may be useful, depending on the pattern of pharmacokinetic decay that the practitioner wishes to achieve. For example, dosing from one-four times a week is contemplated. In some embodiments, dosing ranging from about 3 µg/mg to about 2 mg/kg (such as about 3 µg/mg, about 10 µg/mg, about 30 µg/mg, about 100 µg/mg, about 300 µg/mg, about 1 mg/kg, and about 2 mg/kg) may be used. In some embodiments, dosing frequency is once every week, every 2 weeks, every 4 weeks, every 5 weeks, every 6 weeks, every 7 weeks, every 8 weeks, every 9 weeks, or every 10 weeks; or once every month, every 2 months, or every 3 months, or longer. The progress of this therapy is easily monitored by conventional techniques and assays. The dosing regimen (including the antibody used) can vary over time.

In some embodiments, for an adult patient of normal weight, doses ranging from about 0.3 to 5.00 mg/kg may be administered. In some examples, the dosage of the anti-S1 antibody described herein can be 10 mg/kg. The particular dosage regimen, i.e.., dose, timing and repetition, will depend on the particular individual and that individual’s medical history, as well as the properties of the individual agents (such as the half-life of the agent, and other considerations well known in the art).

For the purpose of the present disclosure, the appropriate dosage of an antibody as described herein will depend on the specific antibody, antibodies, and/or non-antibody peptide (or compositions thereof) employed, the type and severity of the disease/disorder, whether the antibody is administered for preventive or therapeutic purposes, previous therapy, the patient’s clinical history and response to the agonist, and the discretion of the attending physician. Typically the clinician will administer an antibody, until a dosage is reached that achieves the desired result. In some embodiments, the desired result is an increase in anti-tumor immune response in the tumor microenvironment. Methods of determining whether a dosage resulted in the desired result would be evident to one of skill in the art. Administration of one or more antibodies can be continuous or intermittent, depending, for example, upon the recipient’s physiological condition, whether the purpose of the administration is therapeutic or prophylactic, and other factors known to skilled practitioners. The administration of an antibody may be essentially continuous over a preselected period of time or may be in a series of spaced dose, e.g., either before, during, or after developing a target disease or disorder.

As used herein, the term “treating” refers to the application or administration of a composition including one or more active agents to a subject, who has a target disease or disorder, a symptom of the disease/disorder, or a predisposition toward the disease/disorder, with the purpose to cure, heal, alleviate, relieve, alter, remedy, ameliorate, improve, or affect the disorder, the symptom of the disease, or the predisposition toward the disease or disorder.

Alleviating a target disease/disorder includes delaying the development or progression of the disease, or reducing disease severity or prolonging survival. Alleviating the disease or prolonging survival does not necessarily require curative results. As used therein, “delaying” the development of a target disease or disorder means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a target disease or disorder includes initial onset and/or recurrence.

Conventional methods, known to those of ordinary skill in the art of medicine, can be used to administer the pharmaceutical composition to the subject, depending upon the type of disease to be treated or the site of the disease. This composition can also be administered via other conventional routes, e.g., administered orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques. In addition, it can be administered to the subject via injectable depot routes of administration such as using 1-, 3-, or 6-month depot injectable or biodegradable materials and methods. In some examples, the pharmaceutical composition is administered intraocularly or intravitreally.

Injectable compositions may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like). For intravenous injection, water soluble antibodies can be administered by the drip method, whereby a pharmaceutical formulation containing the antibody and a physiologically acceptable excipient is infused. Physiologically acceptable excipients may include, for example, 5% dextrose, 0.9% saline, Ringer’s solution or other suitable excipients. Intramuscular preparations, e.g., a sterile formulation of a suitable soluble salt form of the antibody, can be dissolved and administered in a pharmaceutical excipient such as Water-for-Injection, 0.9% saline, or 5% glucose solution.

In one embodiment, an antibody is administered via site-specific or targeted local delivery techniques. Examples of site-specific or targeted local delivery techniques include various implantable depot sources of the antibody or local delivery catheters, such as infusion catheters, an indwelling catheter, or a needle catheter, synthetic grafts, adventitial wraps, shunts and stents or other implantable devices, site specific carriers, direct injection, or direct application. See, e.g., PCT Publication No. WO 00/53211 and U.S. Pat. No. 5,981,568.

Targeted delivery of therapeutic compositions containing an antisense polynucleotide, expression vector, or subgenomic polynucleotides can also be used. Receptor-mediated DNA delivery techniques are described in, for example, Findeis et al., Trends Biotechnol. (1993) 11:202; Chiou et al., Gene Therapeutics: Methods And Applications Of Direct Gene Transfer (J. A. Wolff, ed.) (1994); Wu et al., J. Biol. Chem. (1988) 263:621; Wu et al., J. Biol. Chem. (1994) 269:542; Zenke et al., Proc. Natl. Acad. Sci. USA (1990) 87:3655; Wu et al., J. Biol. Chem. (1991) 266:338.

Therapeutic compositions containing a polynucleotide (e.g., those encoding the antibodies described herein) are administered in a range of about 100 ng to about 200 mg of DNA for local administration in a gene therapy protocol. In some embodiments, concentration ranges of about 500 ng to about 50 mg, about 1 µg to about 2 mg, about 5 µg to about 500 µg, and about 20 µg to about 100 µg of DNA or more can also be used during a gene therapy protocol.

The therapeutic polynucleotides and polypeptides described herein can be delivered using gene delivery vehicles. The gene delivery vehicle can be of viral or non-viral origin (see generally, Jolly, Cancer Gene Therapy (1994) 1:51; Kimura, Human Gene Therapy (1994) 5:845; Connelly, Human Gene Therapy (1995) 1:185; and Kaplitt, Nature Genetics (1994) 6:148). Expression of such coding sequences can be induced using endogenous mammalian or heterologous promoters and/or enhancers. Expression of the coding sequence can be either constitutive or regulated.

Viral-based vectors for delivery of a desired polynucleotide and expression in a desired cell are well known in the art. Exemplary viral-based vehicles include, but are not limited to, recombinant retroviruses (see, e.g., PCT Publication Nos. WO 90/07936; WO 94/03622; WO 93/25698; WO 93/25234; WO 93/11230; WO 93/10218; WO 91/02805; U.S. Pat. Nos. 5,219,740 and 4,777,127; GB Patent No. 2,200,651; and EP Patent No. 0 345 242), alphavirus-based vectors (e.g., Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532)), and adeno-associated virus (AAV) vectors (see, e.g., PCT Publication Nos. WO 94/12649, WO 93/03769; WO 93/19191; WO 94/28938; WO 95/11984 and WO 95/00655). Administration of DNA linked to killed adenovirus as described in Curiel, Hum. Gene Ther. (1992) 3:147 can also be employed.

Non-viral delivery vehicles and methods can also be employed, including, but not limited to, polycationic condensed DNA linked or unlinked to killed adenovirus alone (see, e.g., Curiel, Hum. Gene Ther. (1992) 3:147); ligand-linked DNA (see, e.g., Wu, J. Biol. Chem. (1989) 264:16985); eukaryotic cell delivery vehicles cells (see, e.g., U.S. Pat. No. 5,814,482; PCT Publication Nos. WO 95/07994; WO 96/17072; WO 95/30763; and WO 97/42338) and nucleic charge neutralization or fusion with cell membranes. Naked DNA can also be employed. Exemplary naked DNA introduction methods are described in PCT Publication No. WO 90/11092 and U.S. Pat. No. 5,580,859. Liposomes that can act as gene delivery vehicles are described in U.S. Pat. No. 5,422,120; PCT Publication Nos. WO 95/13796; WO 94/23697; WO 91/14445; and EP Patent No. 0524968. Additional approaches are described in Philip, Mol. Cell. Biol. (1994) 14:2411, and in Woffendin, Proc. Natl. Acad. Sci. (1994) 91:1581.

The particular dosage regimen, i.e.., dose, timing and repetition, used in the method described herein will depend on the particular subject and that subject’s medical history.

In some embodiments, more than one antibody, or a combination of an antibody and another suitable therapeutic agent, may be administered to a subject in need of the treatment. The antibody can also be used in conjunction with other agents that serve to enhance and/or complement the effectiveness of the agents.

Treatment efficacy for a target disease/disorder can be assessed by methods well-known in the art.

### Diagnostic Applications

Any of the anti-S1 antibodies disclosed herein also can be used for detecting presence of the S protein of SARS-CoV2 or the SARS-CoV2 virus in a sample. In some instances, the sample can be a biological sample such as a blood sample obtained from a subject (e.g., a human subject) suspected of having SARS-CoV2 infection.

To perform the method disclosed herein, any of the anti-S1 antibodies disclosed herein can be brought in contact with a sample suspected of containing a SARS-CoV2 virus or the S protein thereof. In general, the term “contacting” or “in contact” refers to an exposure of the anti-S 1 antibody disclosed herein with the sample suspected of containing the target antigen for a suitable period under suitable conditions sufficient for the formation of a complex between the anti-S1 antibody and the target antigen in the sample, if any. In some embodiments, the contacting is performed by capillary action in which a sample is moved across a surface of the support membrane. The antibody-antigen complex thus formed, if any, can be determined via a routine approach. Detection of such an antibody-antigen complex after the incubation is indicative of the presence of the target antigen in the sample. When needed, the amount of the antibody-antigen complex can be quantified, which is indicative of the level of the target antigen in the sample.

In some embodiments, a target antigen disclosed herein (i.e., the SARS-CoV2 virus or the S protein thereof) in a sample can be detected or quantified using any of the anti-S1 antibodies disclosed herein via an immunoassay. Examples of immunoassays include, without limitation, immunoblotting assay (e.g., Western blot), immunohistochemical analysis, flow cytometry assay, immunofluorescence assay (IF), enzyme linked immunosorbent assays (ELISAs) (e.g., sandwich ELISAs), radioimmunoassays, electrochemiluminescence-based detection assays, magnetic immunoassays, lateral flow assays, and related techniques. Additional suitable immunoassays for detecting the target antigen in a sample will be apparent to those of skill in the art.

In some examples, the anti-S1 antibodies as described herein can be conjugated to a detectable label, which can be any agent capable of releasing a detectable signal directly or indirectly. The presence of such a detectable signal or intensity of the signal is indicative of presence or quantity of the target antigen in the sample. Alternatively, a secondary antibody specific to the anti-S1 or specific to the target antigen may be used in the methods disclosed herein. For example, when the anti-S1 antibody used in the method is a full-length antibody, the secondary antibody may bind to the constant region of the anti-S 1 antibody. In other instances, the secondary antibody may bind to an epitope of the target antigen that is different from the binding epitope of the anti-S1 antibody. Any of the secondary antibodies disclosed herein may be conjugated to a detectable label.

Any suitable detectable label known in the art can be used in the assay methods described herein. In some embodiments, a detectable label can be a label that directly releases a detectable signal. Examples include a fluorescent label or a dye. A fluorescent label comprises a fluorophore, which is a fluorescent chemical compound that can re-emit light upon light excitation. Examples of fluorescent label include, but are not limited to, xanthene derivatives (e.g., fluorescein, rhodamine, Oregon green, eosin, and Texas red), cyanine derivatives (e.g., cyanine, indocarbocyanine, oxacarbocyanine, thiacarbocyanine, and merocyanine), squaraine derivatives and ring-substituted squaraines (e.g., Seta and Square dyes), squaraine rotaxane derivatives such as SeTau dyes, naphthalene derivatives (e.g., dansyl and prodan derivatives), coumarin derivatives, oxadiazole derivatives (e.g., pyridyloxazole, nitrobenzoxadiazole and benzoxadiazole), anthracene derivatives (e.g., anthraquinones, including DRAQ5, DRAQ7 and CyTRAK Orange), pyrene derivatives such as cascade blue, oxazine derivatives (e.g., Nile red, Nile blue, cresyl violet, and oxazine 170), acridine derivatives (e.g., proflavin, acridine orange, and acridine yellow), arylmethine derivatives (e.g., auramine, crystal violet, and malachite green), and tetrapyrrole derivatives (e.g., porphin, phthalocyanine, and bilirubin). A dye can be a molecule comprising a chromophore, which is responsible for the color of the dye. In some examples, the detectable label can be fluorescein isothiocyanate (FITC), phycoerythrin (PE), biotin, Allophycocyanin (APC) or Alexa Fluor® 488.

In some embodiments, the detectable label may be a molecule that releases a detectable signal indirectly, for example, via conversion of a reagent to a product that directly releases the detectable signal. In some examples, such a detectable label may be an enzyme (e.g., β-galactosidase, HRP or AP) capable of producing a colored product from a colorless substrate.

### Kits for Use in Treatment of COVID-19 or Detecting SARS-CoV2 Infection

The present disclosure also provides kits for use in treating or alleviating a target disease, such as SARS-CoV-2 infection or COVID-19 as described herein. The present disclosure also provides kits for use in detecting presence of SARS-CoV2 or S protein thereof in a sample. Such kits can include one or more containers comprising an anti-S1 antibody, e.g., any of those described herein. In some instances, the anti-S1 antibody may be co-used with a second therapeutic agent.

In some embodiments, the kit can comprise instructions for use in accordance with any of the methods described herein. The included instructions can comprise a description of administration of the anti-S1 antibody, and optionally the second therapeutic agent, to treat, delay the onset, or alleviate a target disease as those described herein. The kit may further comprise a description of selecting an individual suitable for treatment based on identifying whether that individual has the target disease, e.g., applying the diagnostic method as described herein. In still other embodiments, the instructions comprise a description of administering an antibody to an individual at risk of the target disease.

The instructions relating to the use of an anti-S1 antibody generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable. The label or package insert indicates that the composition is used for inhibiting SARS-CoV2 infection or treating COVID-19.

Alternatively, the kit can comprise a description of detecting or quantifying SARS-CoV2 or S protein thereof in a sample as disclosed herein. Instructions supplied in the kits of the invention are typically written instructions on a label or package insert (e.g., a paper sheet included in the kit), but machine-readable instructions (e.g., instructions carried on a magnetic or optical storage disk, or available via an internet address provided in the kit) are also acceptable.

The kits disclosed herein are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is an anti-S1 antibody as those described herein.

Kits may optionally provide additional components such as buffers and interpretive information. Normally, the kit comprises a container and a label or package insert(s) on or associated with the container. In some embodiments, the invention provides articles of manufacture comprising contents of the kits described above.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introuction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D.N. Glover ed. 1985); Nucleic Acid Hybridization (B.D. Hames & S.J. Higgins eds.(1985»; Transcription and Translation (B.D. Hames & S.J. Higgins, eds. (1984»; Animal Cell Culture (R.I. Freshney, ed. (1986»; Immobilized Cells and Enzymes (1RL Press, (1986»; and B. Perbal, A practical Guide To Molecular Cloning (1984); F.M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

Sequence of SARS-CoV-2
>SARS-CoV-2 Spike Protein
MFVFLVLLPLVSSQCVNLTTRTQLPPAYTNSFTRGVYYPDKVFRSSVLHS
TQDLFLPFFSNVTWFHAIHVSGTNGTKRFDNPVLPFNDGVYFASTEKSNI
IRGWIFGTTLDSKTQSLLIVNNATNVVIKVCEFQFCNDPFLGVYYHKNNK
SWMESEFRVYSSANNCTFEYVSQPFLMDLEGKQGNFKNLREFVFKNIDGY
FKIYSKHTPINLVRDLPQGFSALEPLVDLPIGINITRFQTLLALHRSYLT
PGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASV
YAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYN
YLYRLFRKSNLKPFERDISTEIYQAGSTPCNGVEGFNCYFPLQSYGFQPT
NGVGYQPYRVVVLSFELLHAPATVCGPKKSTNLVKNKCVNFNFNGLTGTG
VLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCL
IGAEHVNNSYECDIPIGAGICASYQTQTNSPRRARSVASQSIIAYTMSLG
AENSVAYSNNSIAIPTNFTISVTTEILPVSMTKTSVDCTMYICGDSTECS
NLLLQYGSFCTQLNRALTGIAVEQDKNTQEVFAQVKQIYKTPPIKDFGGF
NFSQILPDPSKPSKRSFIEDLLFNKVTLADAGFIKQYGDCLGDIAARDLI
CAQKFNGLTVLPPLLTDEMIAQYTSALLAGTITSGWTFGAGAALQIPFAM
QMAYRFNGIGVTQNVLYENQKLIANQFNSAIGKIQDSLSSTASALGKLQD
VVNQNAQALNTLVKQLSSNFGAISSVLNDILSRLDKVEAEVQIDRLITGR
LQSLQTYVTQQLIRAAEIRASANLAATKMSECVLGQSKRVDFCGKGYHLM
SFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKE
ELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSC
GSCCKFDEDDSEPVLKGVKLHYT
(SEQ ID NO:48)
>SARS-CoV-2-RBD (328-533)-Fc
RFPNITNLCPFGEVFNATRFASVYAWNRKRISNCVADYSVLYNSASFSTF
KCYGVSPTKLNDLCFTNVYADSFVIRGDEVRQIAPGQTGKIADYNYKLPD
DFTGCVIAWNSNNLDSKVGGNYNYLYRLFRKSNLKPFERDISTEIYQAGS
TPCNGVEGFNCYFPLQSYGFQPTNGVGYQPYRVVVLSFELLHAPATVCGP
KKSTNLDKTHTCPPCPAPELLGGPSVFLFPPKPKDTLMISRTPEVTCVVV
DVSHEDPEVKFNWYVDGVEVHNAKTKPREEQYNSTYRWSVLTVLHQDWLN
GKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKS
RWQQGNVFSCSVMHEALHNHYTQKSLSLSPGK
(SEQ ID NO:49)
>SARS-CoV-2-RBD (328-533)-His
RFPNITNLCPFGEVFNATRFASVYAWNRKRISNCVADYSVLYNSASFSTF
KCYGVSPTKLNDLCFTNVYADSFVIRGDEVRQIAPGQTGKIADYNYKLPD
DFTGCVIAWNSNNLDSKVGGNYNYLYRLFRKSNLKPFERDISTEIYQAGS
TPCNGVEGFNCYFPLQSYGFQPTNGVGYQPYRVWLSFELLHAPATVCGPK
KSTNLGSHHHHHH-
(SEQ ID NO:50)

### Example 1 Screening for anti-SARS-CoV-2 Antibodies

Natural human antibody libraries were constructed from multiple donors of bone marrow mononuclear cells (MNCs) and peripheral blood mononuclear cells (PBMCs) of naïve health donors and autoimmune disease patients. RT-PCR was used to capture the full immunoglobulin repertoire of both VH and VL domains. A scFv library was then constructed by V_{H} and V_{L} shuffling. The library size was predicted to be 10¹²⁻¹³. The scFv libraries were further modified to have in vitro transcription and translation signal at N terminus and a flag tag was added to the C-terminus for selection with mRNA display.

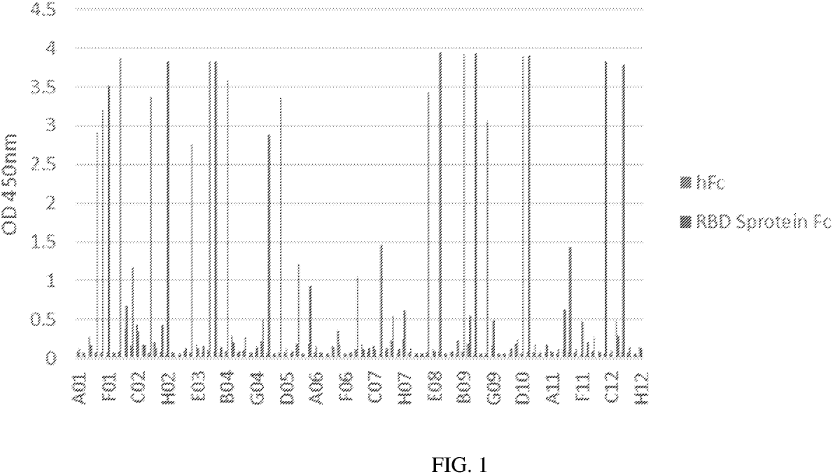

mRNA display technology was then used for the identification of SARS-CoV-2 S1 protein- and RBD-binders with the above constructed scFv library (FIG. 1). Briefly, the DNA library was first transcribed into a mRNA library and then translated into a mRNA-scFv fusion library by covalent coupling through a puromycin linker. The library was purified and converted to a mRNA/cDNA fusion library similar to a known procedure (U.S. Pat. No. 6,258,558, the relevant disclosures of which are incorporated by reference for the subject matter and purpose referenced therein). The mRNA display libraries were first counter-selected with human and mouse IgGs (negative proteins) to remove nonspecific binders, followed by selection against either recombinant RBD-Fc or S1-Fc proteins in solution and then captured with Protein G magnetic beads. The binders were eluted off either by pH stripping to recover all binders or by epitope-directed elution with recombinant ACE2 to recover binders that are potentially block RBD and ACE2 interaction. The RBD and S1 binders were recovered and enriched by PCR amplification each round. Five rounds of selections and enrichment were completed with each target before screening.

### Example 2 Antibody Binding to RBD and S1 Proteins of SARS-CoV-2

After the five rounds of selection described above, enriched libraries were cloned into bacterial periplasmic expression vector pET22b and transformed into TOP10 competent cells. Each scFv molecule was engineered to have a C-terminal flag and a 6xHIS tag for purification and assay detection. Clones from TOP10 cells were pooled and the miniprep DNA was prepared and subsequently transformed into bacterial Rosetta II strain for expression. Single clones were picked, grown and induced with 0.1 mM IPTG in 96 well plate for expression. The supernatant was collected after induction at 30° C. for 16-24 hours.

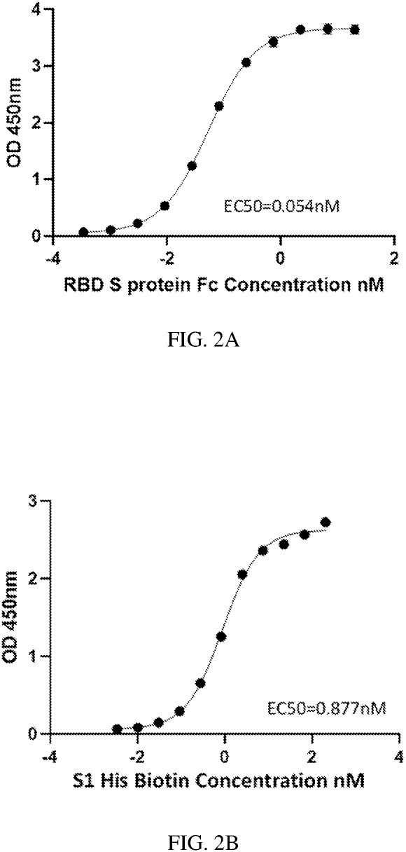

RBD and S1 binding screening ELISA was developed for the identification of individual anti-RBD and S1 scFv, respectively. Briefly. Briefly, a 384-well plate was immobilized with human Fc and human RBD or S1 protein, respectively, at final concentration of 2 µg/mL in 1x PBS in total volume of 25 µL per well. The plate was incubated overnight at 4° C. followed by blocking with 80 µL of superblock per well for 1 hour. 50 µL of supernatant was added to both Fc and RBD- or S1- immobilized wells and incubated for 1 hour with shaking. The RBD (FIG. 2A) or S1 (FIG. 2B) binding activity was detected by adding 25 µL of anti-Flag HRP diluted at 1:5000 in 1x PBST. In between each step, the plate was washed 3 times with 1XPBST in a plate washer. The plate was then developed with 25 µL of TMB substrate for 5 mins and stopped by adding 25 µL of 2N sulfuric acid. The plate was read at OD450 nm with a BIOTEK plate reader and the binding and selectivity was analyzed with an Excel bar graph.

### Example 3 Binding of RBD and S1 Proteins of SARS-CoV-2 to ACE2 in Cells

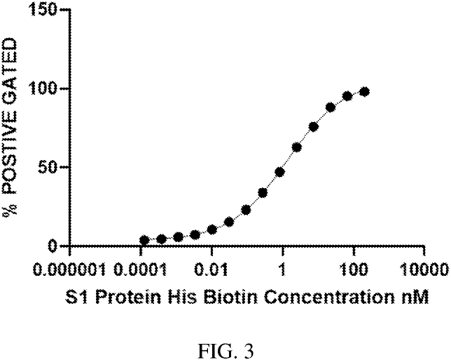

An ACE2 recombinant cell line was generated by transducing an ACE2 lentivirus construct into CHO-K1 cells, followed by G418 drug selection. Cells expressing high levels of ACE2 were sorted and used for cellular neutralization assays. Recombinant RBD and S1 proteins were directly conjugated to Alexa Fluor 647. The EC50 of S1-protein binding to ACE2-expressing CHO-K1 cells was determined (FIG. 3). Positive binding cells counts were counted and plotted in Prism 8.1 software.

### Example 4 Antibody Expression and Purification, and Determination of Tm

Specific anti-SARS-CoV-2 ScFv clones were picked from a glycerol stock plate and grown overnight into a 5 mL culture in a Thomson 24-well plate with a breathable membrane. This culture, and all subsequent cultures described below were grown at 37° C. and shaking at 225 RPM in Terrific Broth Complete plus 100 µg/mL carbenicillin and 34 µg/mL chloramphenicol, with 1:5000 dilution of antifoam-204 also added, unless specified otherwise. This overnight starter culture was then used to inoculate the larger culture, by adding a 1:100 dilution of starter culture into the designated production culture (50 mL culture in a 125 mL Thomson Ultra Yield flask, 100 mL culture in 250 mL Ultra Yield Thomson flask or 250 mL culture in 500 mL Ultra Yield Thomson flask) and grown until the OD₆₀₀ was 0.5-0.8. At this point, the cultures were induced with a final concentration of IPTG at 0.25 mM and incubated overnight at 30° C. The following day, the cultures were spun for 1 hour at 5000 x g, to pellet the cells.

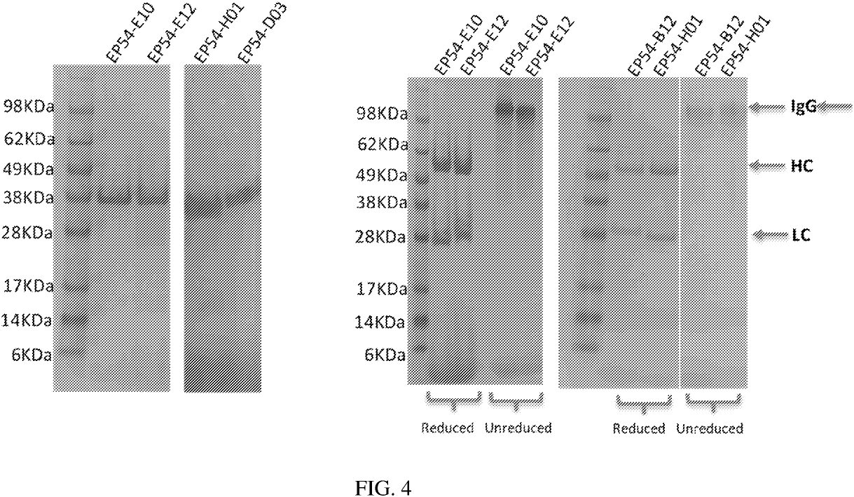

For the purification, 3 µL GE Ni Sepharose Excel resin per 1 mL of supernatant was used. Disposable 10 mL or 20 mL BioRad Econo-Pac columns were used. The resin was equilibrated with at least 20 column volume (CV) buffer A (1xPBS, pH7.4). The filter-sterilized supernatant was purified by gravity flow by either controlling the flow to 1 mL/min or was poured over two times, over the same packed resin bed. The column was then washed with the following buffers: 10 CV buffer A, 20 CV buffer B (1xPBS, pH7.4 and 20 mM imidazole). For 250 mL expression culture purifications, antibody-bound columns were washed sequentially with 20 CV buffer A and 20 CV buffer B. The proteins were eluted with Eluting buffer C (1xPBS pH 7.4, and 500 mM imidazole). Fractions were run on a Bradford assay (100 µL diluted Bradford solution + 10 µL sample). Fractions with bright blue color were pooled. Protein concentration was measured by A280 extension coefficient, and a SDS-PAGE gel was used to analyze the purity of the purified antibodies (FIG. 4). In some cases, ScFv antibodies were further purified by a flag tag affinity purification following the standard protocol. Most of the purified ScFv have >90% purity with reasonable yield.

Selected scFv antibodies were converted into IgG1 format. Specifically, the variable regions of heavy chain and light chain were amplified by PCR and subsequently assembled into the framework of human IgG1 in the vector pCDNA3.4. The constructs were sequence-confirmed before antibody production in mammalian cells. Antibodies were then expressed transiently in ExpiHEK293-F cells in free style system (Invitrogen) according to standard protocol. The cells were grown in above conditions for 7 days before harvesting. The supernatant was collected by centrifugation and filtered through a 0.2 µm PES membrane. The antibodies were purified by MabSelect PrismA protein A resin (GE Health). The protein was eluted with 100 mM Gly pH2.5 + 150 mM NaCl and quickly neutralized with 20 mM citrate pH 5.0 + 300 mM NaCl. Antibodies were concentrated and buffer exchanged to 1xPBS, pH 7.4. The purified IgG1 antibodies had >90% purity, as detected by SDS-PAGE analysis.

For thermostability analysis, each sample and control were prepared in at least a duplicate to make sure the results were reproducible. A plate map was designed first in Excel so the exact location of each sample could be matched to the software for running and analyzing the samples. A fresh dilution of Protein Thermal Shift Dye (1000x) to 8x was prepared in water. A MicroAmp Optical 96 well plate or 8 cap strip by LifeTech was used for the experiments. The following reagents were added in the order listed: 1st: 5 µL Protein Thermal Shift Buffer, 2nd: sample: 12.5 µL sample diluted to 0.4 mg/mL in water, no protein in 12.5 µL buffer was used as negative control and 10.5 µL water with 2.0 µL Protein Thermal Shift Control Protein as positive control, 3rd: 2.5 µL diluted Thermal Shift Dye 8x for a total volume of 20 µL/well. The Thermal shift dye once added, was pipetted up and down 10x. The plates or strips were then spun down for 1000 RPM for 1 min once sealed with MicroAmp Optical film of caps. The plate or strips were then put into a Quant Studio 3 instrument by Thermo Fisher with the proceeding method being run. Step 1: 100% ramp rate to 25.0° with time 2 min and finally Step 2: 1% ramp rate to 99.0° C. with time 2 min. The samples and subsequent Tm were then analyzed (and Tm calculated) using the QuantStudio Design and Analysis Software and the Protein Thermal Shift Software 1.3. The examples of Tm for scFv antibodies are shown in Table 1 below.

TABLE 1

Melting Temperatures for Exemplary scFv Antibodies

| ScFv | Tm (°C) |
|---|---|
| 2020EP53-D06 | 69.3 |
| 2020EP54-H01 | 55.1 |
| 2020EP54-E12 | 55.3 |
| 2020EP54-B02 | 63.6 |
| 2020EP54-E10 | 61.8 |
| 2020EP60-F05 | 72.9 |
| 2020EP66-A07 | 59.7 |
| 2020EP64-C08 | 54.5 |

### Example 5 Binding of scFv Antibodies to RBD and S1 Proteins of SARS-CoV-2 Using ELISA

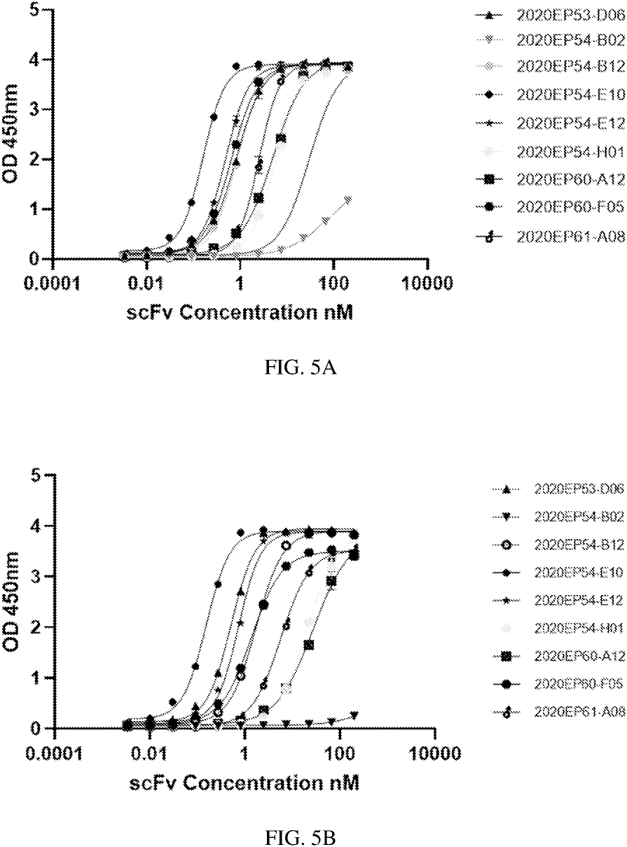

An ELISA assay was developed to determine the EC50 of anti-RBD and S1 antibodies. Briefly, a 384 well plate was immobilized with human RBD or S1 at final concentration of 2 ug/mL in 1x PBS in total volume of 25 µL per well. The plate was incubated overnight at 4° C. followed by blocking with 80 µL of superblock per well for 1 hour. Purified anti-RBD or Anti-S1 scFvs were 2-fold serial diluted from 200 nM to 0 for 16 points. 25 µL was added to RBD- or S1-immobilized wells and incubated for 1 hour with shaking. The RBD or S1 binding was detected by adding 25 µL of anti-Flag HRP diluted at 1:5000 in 1x PBST. In between each step, the plate was washed 3 times with 1XPBST in a plate washer. The plate was then developed with 20 ul of TMB substrate for 5 mins and stopped by adding 20 ul of 2N sulfuric acid. The plate was read at OD450 nm Biotek plate reader and then plotted in Prism 8.1 software. The binding of examples of scFv antibodies to RBD (FIG. 5A) and S1 (FIG. 5B) were tested using ELISA assays.

The EC50 of scFv binding to RBD are shown in Table 2 below (NB=no binding).

TABLE 2

EC50 of Exemplary scFv Antibody Binding to RBD

| ScFv | Binding to RBD of SARS-COV-2 in ELISA EC50 (nM) | Binding to S1 protein of SARS-COV-2 in ELISA EC50 (nM) |
|---|---|---|
| 2020EP53-D06 | 0.8184 | 0.5115 |
| 2020EP54-H01 | 5.773 | 20.41 |
| 2020EP54-E12 | 0.4857 | 0.7367 |
| 2020EP54-B12 | 0.8526 | 2.683 |
| 2020EP54-B02 | 75.93 | NB |
| 2020EP54-E10 | 0.1602 | 0.1542 |
| 2020EP60-F05 | 0.6494 | 1.378 |
| 2020EP60-A12 | 4.989 | 27.34 |
| 2020EP61-A08 | 2.515 | 5.743 |
| 2020EP61-C12 | 1.439 | 37.72 |
| 2020EP64-G10 | 0.6626 | 0.9365 |
| 2020EP66-D03 | NB | 0.5145 |
| 2020EP64-C08 | 8.732 | 40.19 |
| 2020EP66-A07 | NB | 0.5249 |
| 2020EP71-E04 | NB | 5.029 |
| 2020EP75-E02 | NB | 0.2248 |

### Example 6 Binding of scFv Antibodies to RBD and S1 Proteins of SARS-CoV-2 in SPR

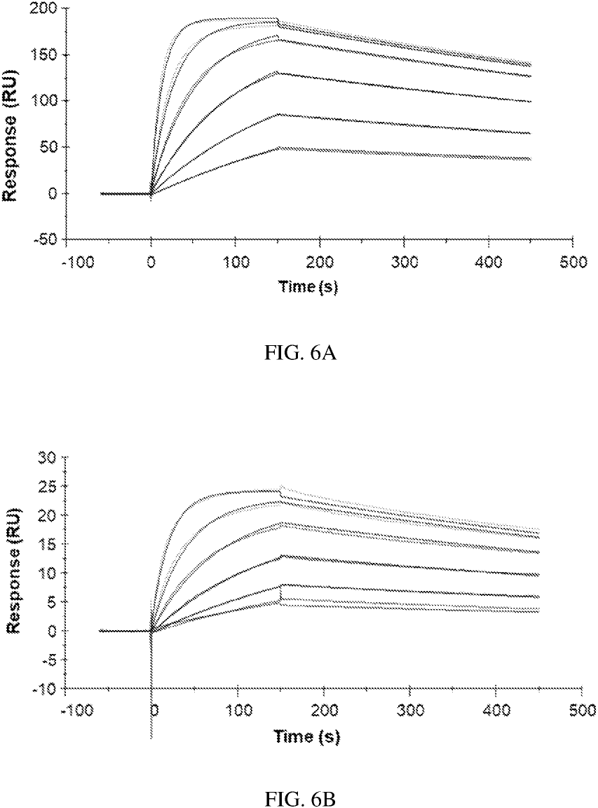

Kinetic analysis of anti-RBD and anti-S1 protein antibodies were assessed by Surface Plasmon Resonance (SPR) technology with a Biacore T200. The assay was run with Biacore T200 control software version 2.0. For each cycle, 1 µg/mL of RBD or S1 protein was captured for 60 seconds at flow rate of 10 µL/min on flow cell 2 in 1XHBSP buffer on anti-human Fc sensor chip. Two-fold serial diluted purified scFv antibodies were injected onto both reference flow cell 1 and a RBD- or S1-protein captured flow cell 2 for 150 seconds at flow rate of 30 µL/mins followed by wash for 300 seconds. The flow cells were then regenerated with Biacore regeneration buffer (3 M MgCl₂) for 30 seconds at flow rate of 30 µL/mins. Eight concentration points from 300-0 nM were assayed per antibody in a 96 well plate. The kinetics data were analyzed with Biacore T200 evaluation software 3000. The specific binding response units were derived from subtraction of binding to reference flow cell 1 from target flow cell 2. FIG. 6 show the sensor gram examples of 2020EP054-E10 binding to RBD (FIG. 6A) and S1 protein (FIG. 6B), respectively. The 2020EP54-E10 scFv binding kinetics are shown in Table 3 below.

TABLE 3

2020EP54-E10 scFv Binding Kinetics

| | Ka (1/Ms) | Kd (1/s) | Kd (M) |
|---|---|---|---|
| RBD | 4.770E+5 | 9.059E-4 | 1.899E-9 |
| S1 | 3.158E+5 | 0.001059 | 3.352E-9 |

The binding kinetics for the scFvs are shown in Table 4 below.

TABLE 4

binding kinetics of Exemplary scFv Antibodies

| | RBD Binding SPR | | | S1 Protein Binding SPR | | |
|---|---|---|---|---|---|---|
| Clone | ka (1/Ms) | kd (1/s) | KD (M) | ka (1/Ms) | kd (1/s) | KD (M) |
| 2020EP53-D06 | 2.51E+05 | 0.001103 | 4.40E-09 | 2.60E+05 | 2.04E-03 | 7.84E-09 |
| 2020EP54-H01 | 8.43E+04 | 2.65E-03 | 3.14E-08 | 9.90E+04 | 5.20E-03 | 5.25E-08 |
| 2020EP54-E12 | 4.89E+04 | 4.69E-04 | 9.61E-09 | 5.19E+04 | 6.87E-04 | 1.32E-08 |
| 2020EP54-B12 | 6.72E+05 | 0.005035 | 7.49E-09 | 5.86E+05 | 0.003834 | 6.54E-09 |
| 2020EP54-B02 | 3.49E+04 | 2.42E-03 | 6.95E-08 | 3.99E+04 | 0.003661 | 9.18E-08 |
| 2020EP54-E10 | 4.77E+05 | 9.06E-04 | 1.89E-09 | 3.158E+5 | 0.001059 | 3.352E-9 |
| 2020EP60-F05 | 4.91E+05 | 3.02E-03 | 6.14E-09 | | | |
| 2020EP61-A08 | 2.75E+04 | 0.001909 | 6.94E-08 | | | |
| 2020EP61-C12 | 9.91E+04 | 0.002799 | 2.82E-08 | | | |
| ACE2-RBD | 7.65E+04 | 3.47E-04 | 4.54E-09 | 1.87E+05 | 2.13E-03 | 1.14E-08 |
| 2020EP64-G10 | | | | 2.15E+05 | 0.002415 | 1.12E-08 |
| 2020EP66-D03 | | | | 1.31E+05 | 0.002561 | 1.95E-08 |
| 2020EP64-C08 | | | | 9.41E+04 | 0.006927 | 7.36E-08 |
| 2020EP66-A07 | | | | 6.14E+05 | 0.001415 | 2.30E-09 |
| 2020EP71-E04 | | | | 5.06E+04 | 0.001903 | 3.76E-08 |
| 2020EP75-E02 | | | | 3.90E+05 | 0.001741 | 4.46E-09 |

### Example 7 Neutralizing Activities of scFvs to RBD and S1 Binding of SARS-CoV-2 to ACE2 in ELISA

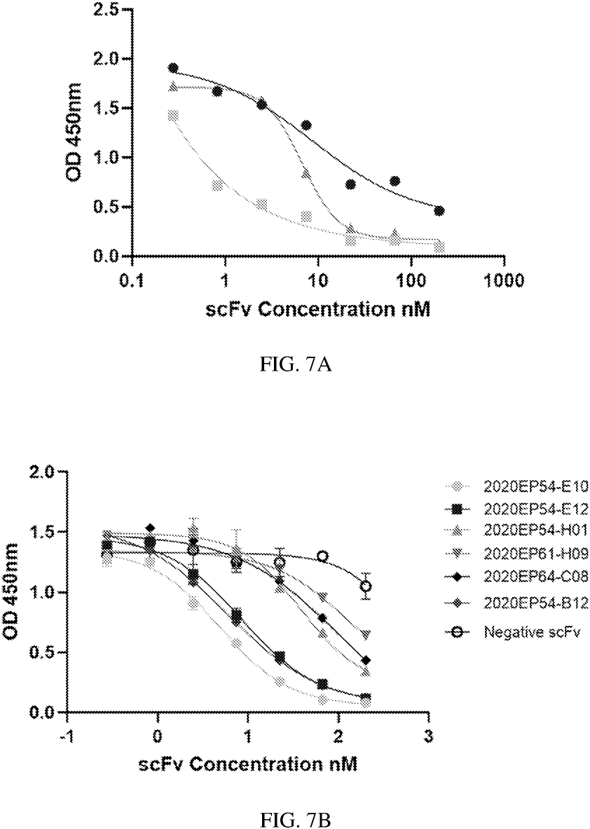

To assess the neutralization activity of the scFv binders, an anti-RBD or S1 competition ELISA with ACE2 was developed. A 384 well plate was immobilized with human ACE2 at a final concentration of 2 µg/mL in 1x PBS in total volume of 25 µL per well. The plate was incubated overnight at 4° C. followed by blocking with 80 µL of superblock per well for 1 hour. 25 µL of serial diluted purified anti-RBD or anti-S 1 scFvs were incubated with EC80 of RBD or S1 for 30 mins and then added to human ACE2 protein immobilized wells, followed by incubation for 1 hour with shaking. The binding activity of RBD or S1 to ACE2 was detected by adding 25 µL of anti-Flag HRP diluted at 1:5000 in 1x PBST. In between each step, the plate was washed 3 times with 1XPBST in a plate washer. The plate was then developed with 20 µL of TMB substrate for 5 mins and stopped by adding 20 µL of 2N sulfuric acid. The plate was read at OD450 nm Biotek plate reader and then plotted in Prism 8.1 software. IC50 was calculated. FIG. 7 shows the neutralization activity examples of RBD (FIG. 7A) and S1 (FIG. 7B) protein interaction with ACE2, respectively.

The IC₅₀ values of binding are shown in Tables 5 and 6 below.

TABLE 5

IC₅₀ values of Exemplary scFvs Binding to RBD-ACE2

| RBD-ACE2 | 2020EP54-B12 | 2020EP054-E10 | 2020EP054-E12 |
|---|---|---|---|
| IC₅₀ (nM) | 8.64 | <1 | 6.69 |

TABLE 6

IC₅₀ values of Exemplary scFvs Binding to S1-ACE2

| S1-ACE2 | 2020EP54-B12 | 2020EP054-E10 | 2020EP054-E12 |
|---|---|---|---|
| IC₅₀ (nM) | 6.05 | 4.94 | 8.57 |
| | 2020EP54-C08 | 2020EP054-H01 | 2020EP054-H09 |
| IC₅₀ (nM) | 94.2 | 38.41118.3 | |

### Example 8 Neutralizing Activities of ScFvs to S1 Binding of SARS-CoV-2 to ACE2/CHOK1 by FACS

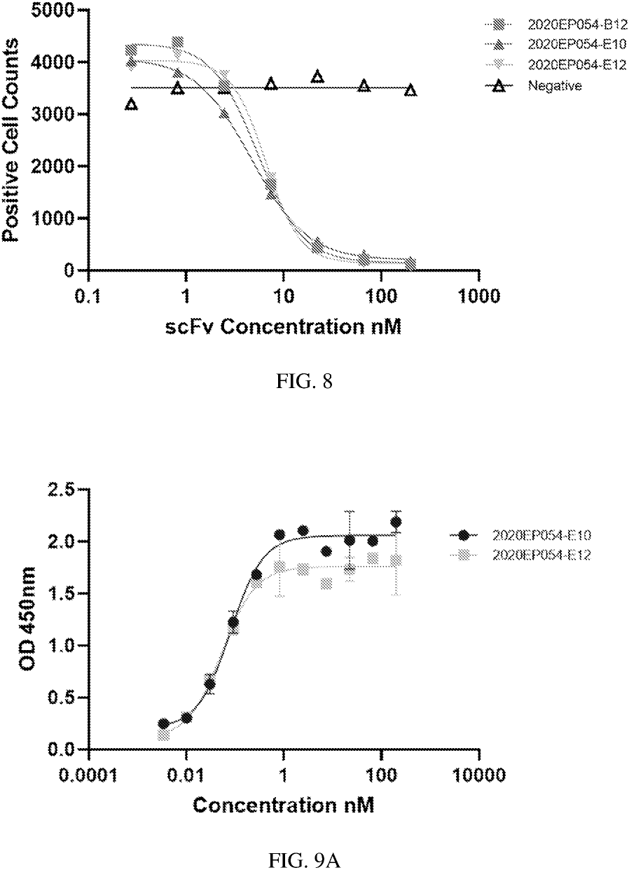

To test anti-RBD or S1 scFv neutralization and blocking human ACE2 cell binding activity, a FACS assay was developed. Briefly, recombinant RBD and S1 were conjugated with AF647. EC50 was determined by binding of serial diluted AF647 conjugated RBD or S1 to a recombinant human ACE2 expressing CHOK1 cell line or an endogenous ACE2-expressing HepG2 cell line. 50 µL of serial diluted purified anti-RBD or anti-S1 scFvs were incubated with EC80 of RBD-AF647 or S1-AF647 for 30 mins and then added to ACE2/CHOK1 or HepG2 cell lines, followed by incubation at 4° C. for 1 hour with shaking. Cells were washed and binding activity of RBD and S1 to human ACE2 cells was detected by Attune flow cytometer, then plotted in Prism 8.1 software. IC50 was calculated. FIG. 8 shows examples of neutralization activities of ScFv antibodies to S1 interaction with ACE2/CHOK1 cells. The IC50 values are shown in Table 7 below.

TABLE 7

IC₅₀ values for Exemplary scFv antibodies

| S1-ACE2/CHKO1 | 2020EP54-E10 | 2020EP054-E12 | 2020EP054-B12 |
|---|---|---|---|
| IC₅₀ (nM) | 4.69 | 6.58 | 5.41 |

### Example 9 Binding of IgG1 Antibodies to RBD and S1 Proteins of SARS-CoV-2 in ELISA

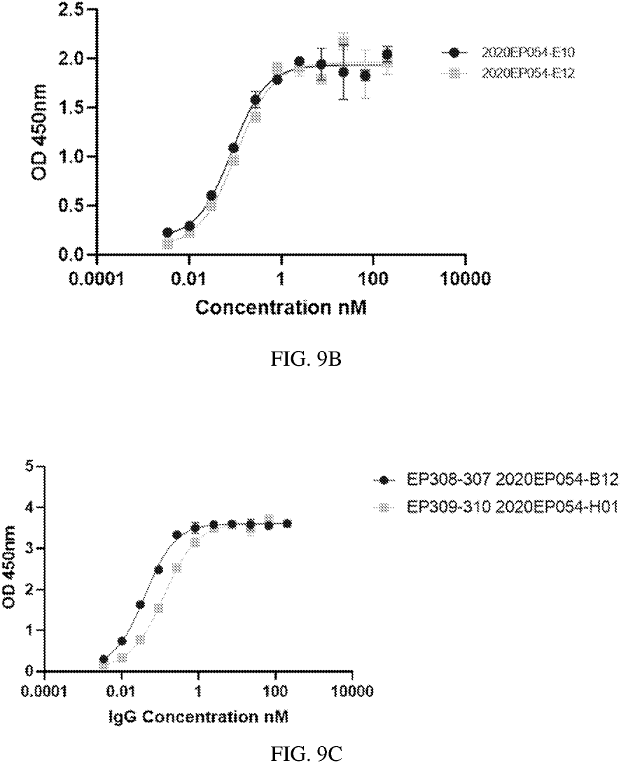

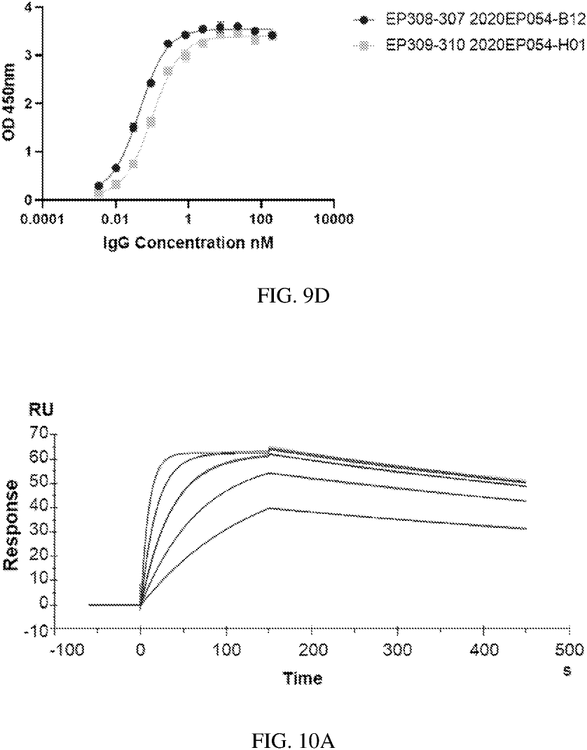

To determine EC50 of anti-RBD or S1 purified IgGs, a 384 well plate was immobilized with RBD or S1 at final concentration of 2 µg/mL in 1x PBS in total volume of 25 µL per well. The plate was incubated overnight at 4° C. followed by blocking with 80 µL of superblock per well for 1 hour. Purified anti-RBD or S1 IgGs were 2-fold serial diluted from 200 nM to 0 for a total of 16 points. 25 µL was added to RBD or S1 immobilized wells and incubated for 1 hour with shaking. The RBD or S1 binding was detected by adding 25 µL of anti-human Fc HRP diluted at 1:10000 in 1x PBST. In between each step, the plate was washed 3 times with 1XPBST in a plate washer. The plate was then developed with 20 µL of TMB substrate for 5 mins and stopped by adding 20 µL of 2N sulfuric acid. The plate was read at OD450 nm Biotek plate reader and then plotted in Prism 8.1 software. FIG. 9 shows the binding activities of IgG antibodies to RBD (FIGS. 9A and C) and S1 (FIGS. 9B and D) protein in ELISA, respectively. The EC50 values are shown in Tables 8 and 9 below.

TABLE 8

EC50 values of Exemplary Antibodies for Binding to RBD

| RBD | 2020EP054-E10 | 2020EP054-E12 | 2020EP054-B12 | 2020EP054-H01 |
|---|---|---|---|---|
| EC50 (nM) | 0.080 | 0.053 | 0.045 | 0.101 |

TABLE 9

EC50 values of Exemplary Antibodies for Binding to S1

| S1 | 2020EP054-E10 | 2020EP054-E12 | 2020EP054-B12 | 2020EP054-H01 |
|---|---|---|---|---|
| EC50 (nM) | 0.084 | 0.103 | 0.045 | 0.101 |

### Example 10 Binding of IgG1 Antibodies to RBD and S1 Proteins of SARS-CoV-2 in SPR

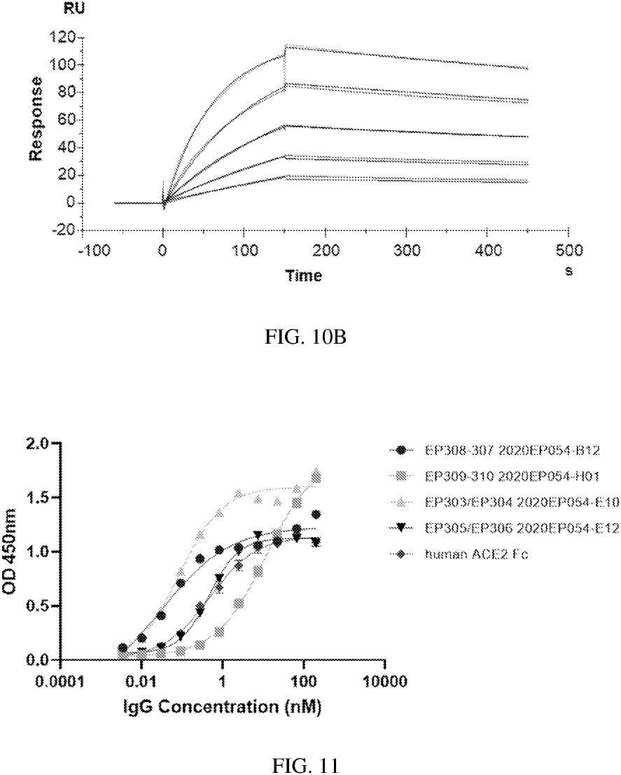

Kinetic analysis of RBD or S1 IgGs were assessed by SPR technology with Biacore T200. The assay was run with Biacore T200 control software version 2.0. A 1:1 kinetics assay was developed to assess SARS-CoV-2 IgGs. Briefly, anti-human Fc was immobilized on CM5 chip to achieve high density. For each cycle, 1 µg/mL of SARS-CoV-2 IgG was captured for 60 seconds at flow rate of 10 ul/min on flow cell 2 in 1xHBSP buffer on anti-human Fc sensor chip. 2-fold serial diluted HIS tagged RBD or S1 was injected onto both reference flow cell 1 and SARS-CoV-2 IgG captured flow cell 2 for 150 seconds at flow rate of 30 µL/mins followed by wash for 300 seconds. The flow cells were then regenerated with anti-human Fc regeneration buffer for 60 seconds at flow rate of 30 µL/mins. Eight concentration points from 300-0 nM were assayed per sample in a 96 well plate. The kinetics of IgG binding to RBD or S1 were analyzed with Biacore T200 evaluation software 3000. The specific binding response unit was derived from subtraction of binding to reference flow cell 1 from IgG captured flow cell 2. FIG. 10 shows examples of sensor grams of IgG antibodies (2020EP54-E10 IgG) to RBD (FIG. 10A) and S1 (FIG. 10B) protein in SPR, respectively. The kinetics parameters are shown in Table 10 below.

TABLE 10

Kinetics parameters of Exemplary IgG Antibody for Binding to RBD and S1

| | | | |
|---|---|---|---|
| | Ka (1/ms) | Kd (1/s) | Kd (M) |
| RBD | 1.456E+6 | 8.070E-4 | 5.544E-10 |
| S1 | 2.169E+5 | 4.980E-4 | 2.296E-9 |

### Example 11 Binding of IgG1 Antibodies to S Proteins of SARS-CoV-2 in ELISA

To determine binding EC50 of anti-RBD or S1 purified IgGs to the S protein of SARS-COV-2, 384 well plate was immobilized with S protein (Sino Biologics) at final concentration of 2 µg/mL in 1x PBS in total volume of 25 µL per well. The plate was incubated overnight at 4° C. followed by blocking with 80 µL of superblock per well for 1 hour. Purified anti-RBD or S1 IgGs were 2-fold serial diluted from 200 nM to 0 for total of 16 points. 25 µL was added to S protein immobilized wells and incubated for 1 hour with shaking. RBD or S1 binding was detected by adding 25 µL of anti-human Fc HRP diluted at 1:10000 in 1x PBST. In between each step, the plate was washed 3 times with 1XPBST in a plate washer. The plate was then developed with 20 µL of TMB substrate for 5 mins and stopped by adding 20 µL of 2N sulfuric acid. The plate was read at OD450 nm Biotek plate reader and then plotted in Prism 8.1 software. FIG. 11 shows binding activities of IgG antibodies to S protein in ELISA.

The EC50 values are shown in Table 11 below.

TABLE 11

EC50 values for Binding Activity of Exemplary IgG antibodies to S protein

| S protein | EP54-E10, IgG1 | EP054-E12, IgG1 | EP054-B12, IgG1 | EP054-H01, IgG1 | hACE2-Fc |
|---|---|---|---|---|---|
| EC50 (nM) | 0.0846 | 0.462 | 0.032 | 11.53 | 0.479 |

### Example 12 Binding of IgG1 Antibodies to RBD and S Proteins of SARS-COV in ELISA

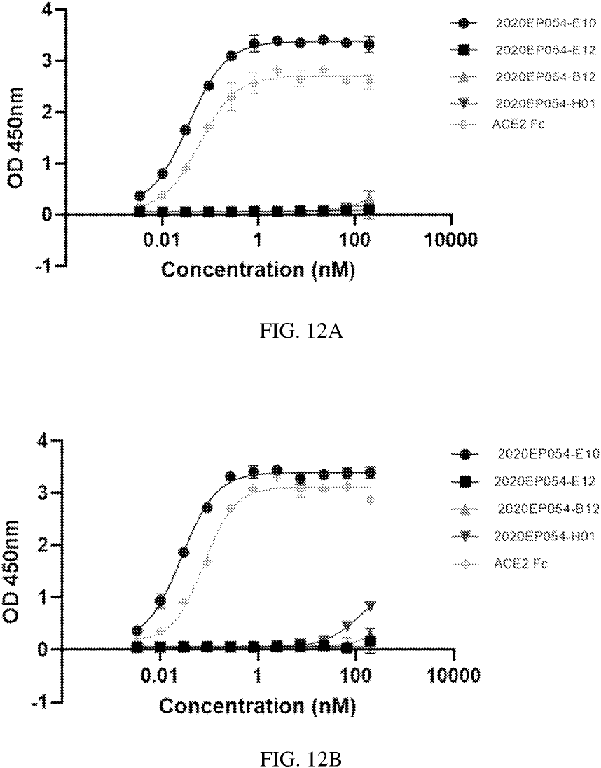

To determine whether the purified IgGs of anti-RBD or S1 protein of SARS-CoV-2 also bind to that of SARS-COV, a 384 well plate was immobilized with RBD or S1 protein of SARS-COV (ACRO Biosciences) at final concentration of 2 µg/mL in 1x PBS in total volume of 25 µL per well. The plate was incubated overnight at 4° C. followed by blocking with 80 µL of superblock per well for 1 hour. Purified anti-RBD or S1 IgGs were 2-fold serial diluted from 200 nM to 0 for a total of 16 points. 25 µL was added to RBD or S1 immobilized wells and incubated for 1 hour with shaking. The RBD or S1 binding was detected by adding 25 µL of anti-human Fc HRP diluted at 1:10000 in 1x PBST. In between each step, the plate was washed 3 times with 1XPBST in a plate washer. The plate was then developed with 20 µL of TMB substrate for 5 mins and stopped by adding 20 µL of 2N sulfuric acid. The plate was read at OD450 nm Biotek plate reader and then plotted in Prism 8.1 software. FIG. 12 shows the binding activities of IgG antibodies to RBD (FIG. 12A) and S1 (FIG. 12B) protein of SARS-COV in ELISA, respectively.

The EC50 values are shown in Tables 12 and 13 below (NB-no binding).

TABLE 12

EC50 values for Binding Activity of Exemplary IgGs to RBD of SARS-COV

| RBD-SARS | 2020EP054 -E10 | 2020EP054-E12 | 2020EP054-B12 | 2020EP054-H01 | ACE2-Fc |
|---|---|---|---|---|---|
| EC50 (nM) | 0.035 | NB | NB | NB | 0.059 |

TABLE 13

EC50 values for binding activity of IgGs to S1 of SARS-COV

| S1-SARS | 2020EP054-E10 | 2020EP054-E12 | 2020EP054-B12 | 2020EP054-H01 | ACE2-Fc |
|---|---|---|---|---|---|
| EC50 (nM) | 0.027 | NB | NB | NB | 0.079 |

### Example 13 Binding of IgG Antibodies to RBD and S1 Proteins of SARS-CoV in SPR

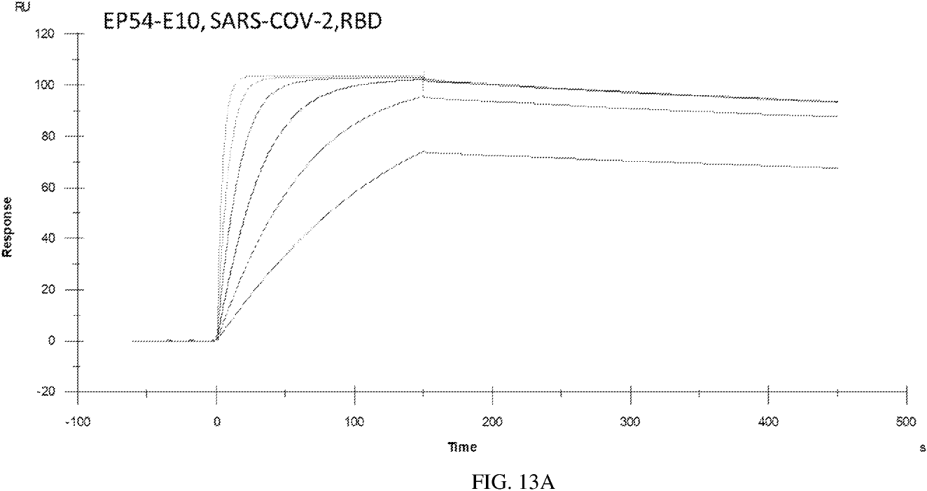

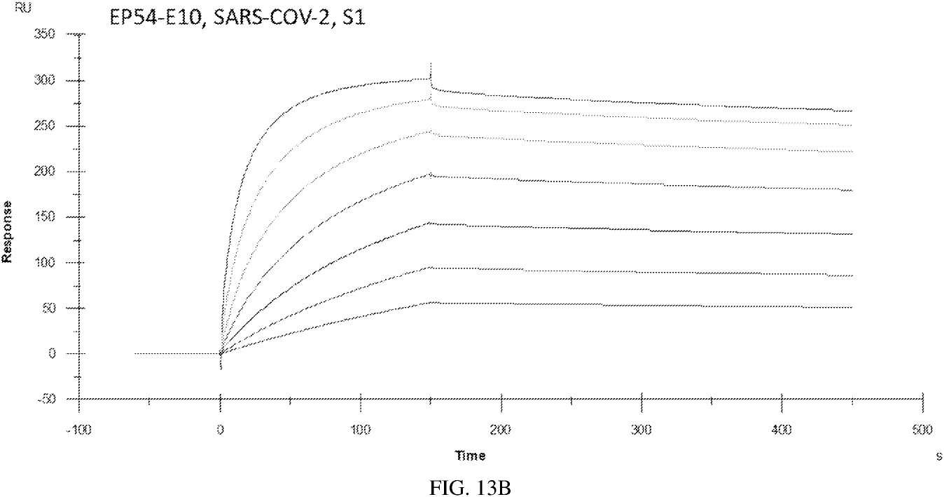

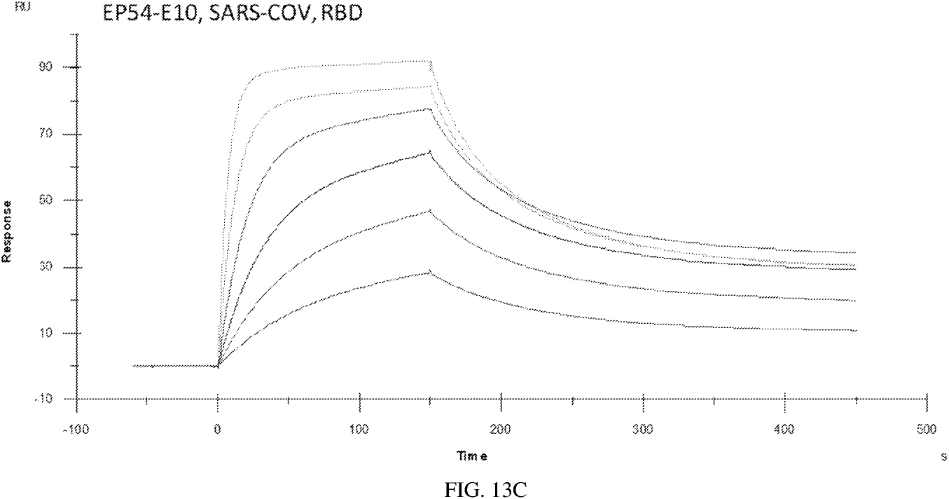

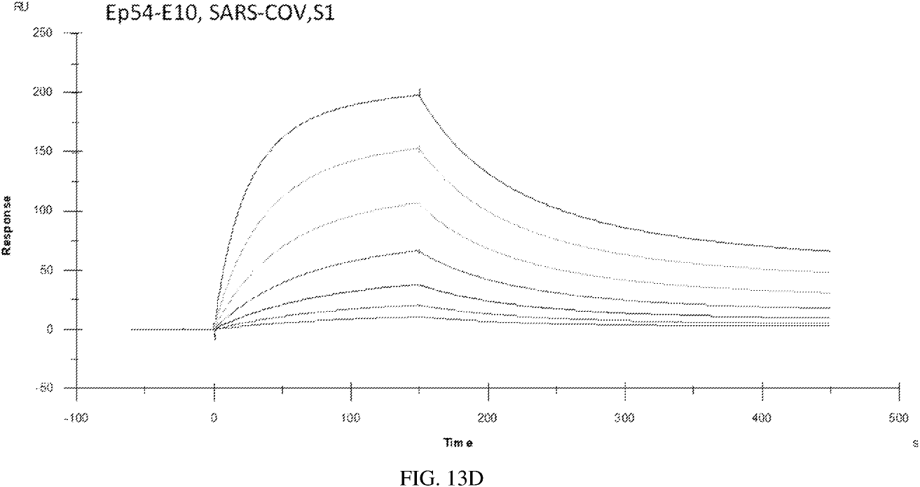

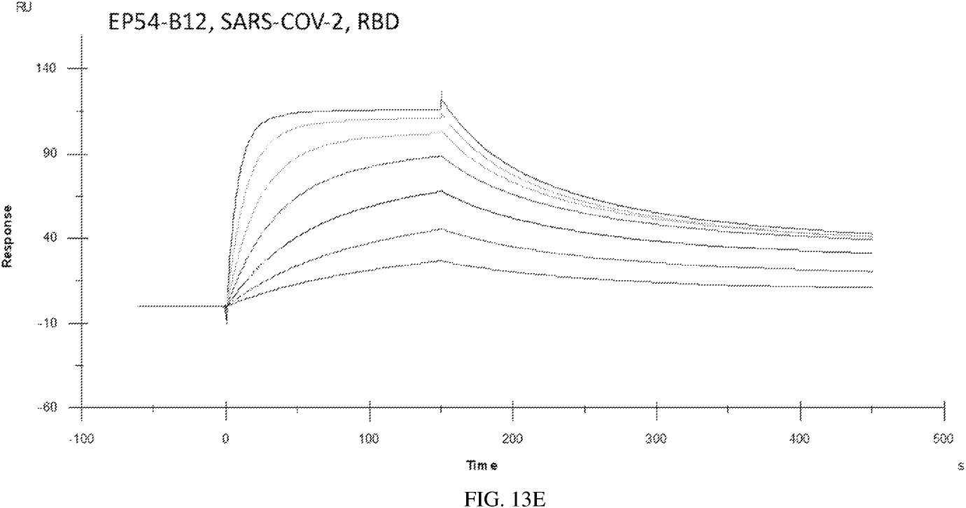

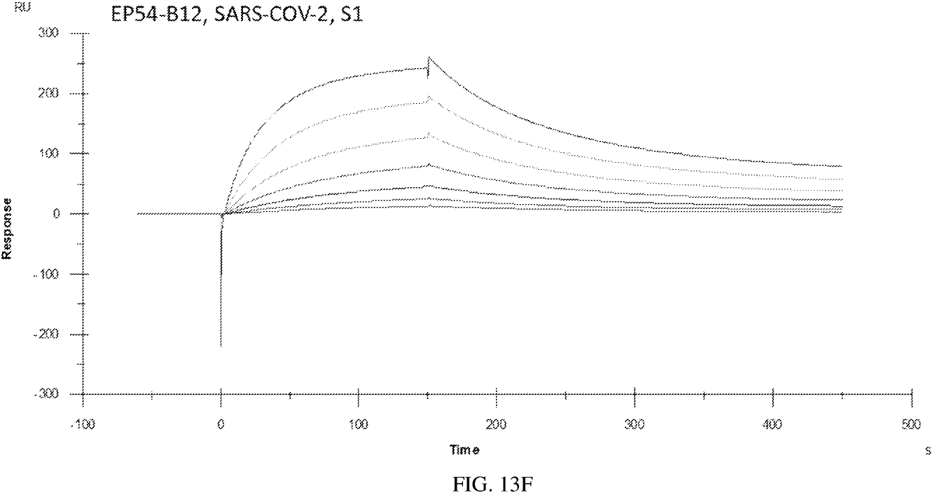

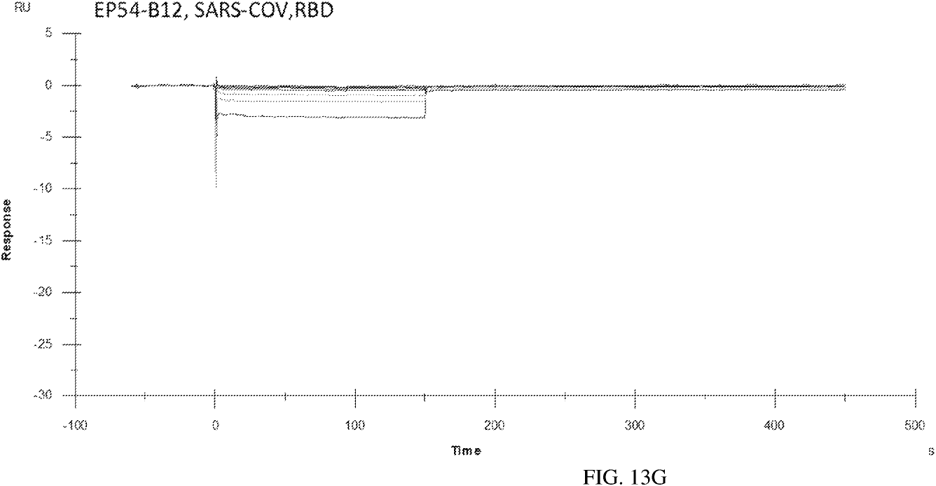

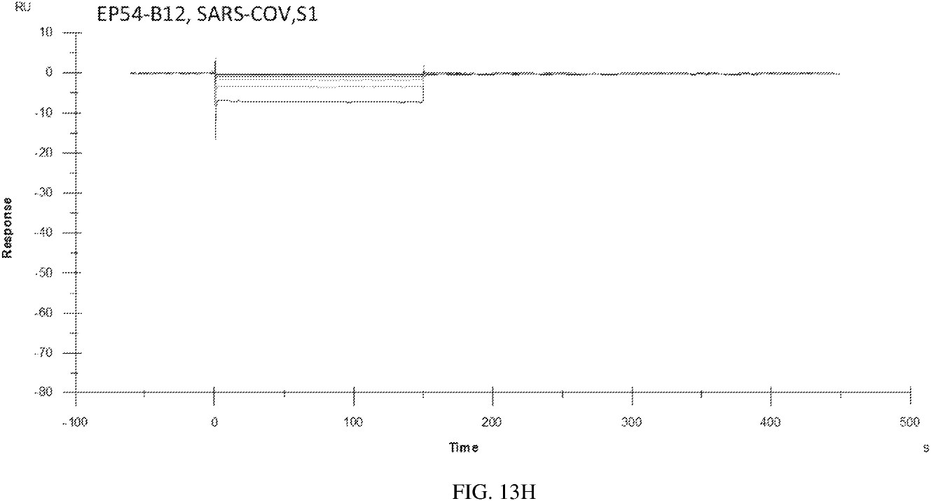

The cross binding kinetic analysis of anti-RBD and anti-S1 protein of SARS-COV-2 antibodies to that of SARS-COV were assessed by Surface Plasmon Resonance (SPR) technology with a Biacore T200. The assay was run with Biacore T200 control software version 2.0. For each cycle, 1 µg/mL of anti-RBD or anti-S1 protein IgG antibodies were captured for 60 seconds at flow rate of 10 µL/min on flow cell 2 in 1XHBSP buffer on protein A sensor chip. Two-fold serial diluted purified RBD or S1 protein of SARS-COV-2 and SARS-COV, respectively were injected onto both reference flow cell 1 and a IgG captured flow cell 2 for 150 seconds at flow rate of 30 µL/mins followed by wash for 300 seconds. The flow cells were then regenerated with glycine pH2.0 buffer for 60 seconds at flow rate of 30 µL/mins. Eight concentration points from 300-0 nM were assayed per antibody in a 96 well plate. The kinetics data were analyzed with Biacore T200 evaluation software 3000. The specific binding response units were derived from subtraction of binding to reference flow cell 1 from target flow cell 2. FIG. 13 show the sensor gram examples of 2020EP054-E10 (FIGS. 13A-D) and 2020EP054-B12 (FIGS. 13E-H) binding to RBD and S1 protein of SARS-COV-2 and SARS-COV, respectively. The binding kinetics of are shown in Table 14 below.

TABLE 14

Binding kinetics of Exemplary Antibodies to RBD and S1 protein of SARS-COV-2 and SARS-COV

| Protein | EP54-E10, IgG | | | EP54-B12, IgG | | |
|---|---|---|---|---|---|---|
| | ka (1/Ms) | kd (1/s) | KD (M) | ka (1/Ms) | kd (1/s) | KD (M) |
| SARS-COV-2, RBD | 2.434E+6 | 3.198E-4 | 1.314E-10 | 9.026E+5 | 5.776E-3 | 6.399E-9 |
| SARS-COV-2, S1 | 2.541E+5 | 2.921E-4 | 1.150E-9 | 8.198E+4 | 5.071E-3 | 6.186E-8 |
| SARS-COV, RBD | 1.828E+6 | 6.748E-3 | 3.691E-9 | *ND | ND | ND |
| SARS-COV, S1 | 7.576E+4 | 4.329E-3 | 5.714E-8 | ND | ND | ND |

### Example 14 Neutralizing Activities of IgG1 Antibodies to RBD and S1 Binding of SARS-CoV-2 to ACE2 in ELISA

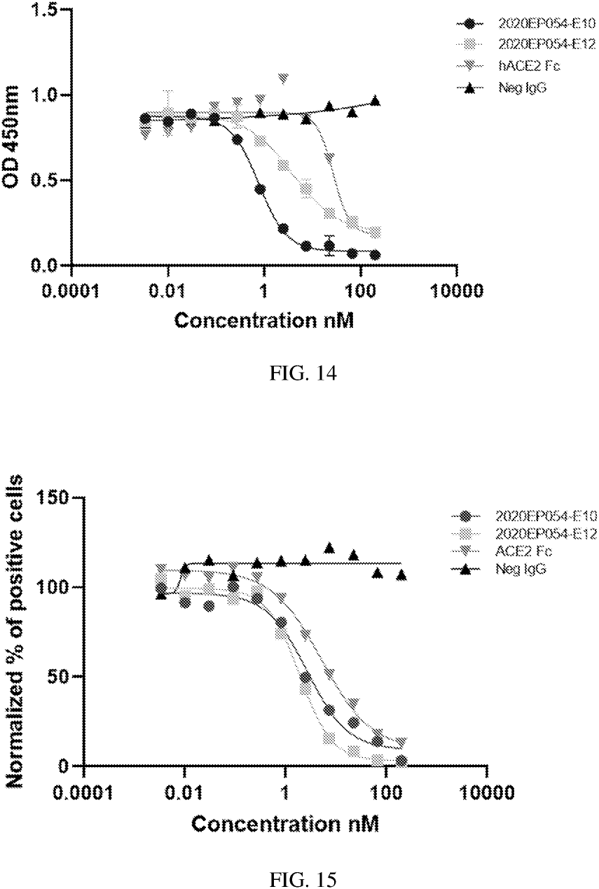

Anti-RBD or S1 IgG neutralization activity was assessed in competition ELISA with RBD or S1 binding to ACE2. Briefly, a 384 well plate was immobilized with human ACE2/His tag at final concentration of 2 µg/mL in 1x PBS in total volume of 25 µL per well. The plate was incubated overnight at 4° C. followed by blocking with 80 µL of superblock per well for 1 hour. 25 µL of serial diluted purified IgGs were mixed with an EC80 concentration of RBD or S1 and incubated for 30 mins then added to ACE2 immobilized wells and incubated for 1 hour with shaking. The RBD or S1 binding was detected by adding 25 µL of anti-human Fc HRP diluted at 1:10000 in 1x PBST. In between each step, the plate was washed 3 times with 1XPBST in a plate washer. The plate was then developed with 20 µL of TMB substrate for 5 mins and stopped by adding 20 µL of 2N sulfuric acid. The plate was read at OD450 nm Biotek plate reader and then plotted in Prism 8.1 software. FIG. 14 shows examples of IgG antibody neutralization of S1 protein binding to ACE2 in ELISA.

The IC50 values are shown in Table 15 below.

TABLE 15

IC50 values of Exemplary Antibodies for Inhibiting S1 Protein Binding to ACE2

| S1 | 2020EP54-E10 | 2020EP054-E12 | hACE2-Fc |
|---|---|---|---|
| IC50 (nM) | 0.774 | 3.79 | 26.98 |

### Example 15 Neutralizing Activities of IgG1 Antibodies to RBD and S1 Binding to ACE2/CHO-K1 by FACS

To test anti-RBD or S1 IgG antibody neutralization and blocking human ACE2 cell binding activity, a FACS assay was developed. Briefly, recombinant RBD and S1 were conjugated with AF647. EC50 was determined by binding of serial diluted AF647 conjugated RBD or S1 to recombinant human ACE2 expressing CHOK1 cell line. 50 µL of serial diluted purified anti-RBD or anti-S1 IgG antibodies was incubated with EC80 of RBD-AF647 or S1-AF647 for 30 mins and then added to human ACE2/CHOK1, followed by incubation at 4° C. for 1 hour with shaking. Cells were washed and binding activity of RBD and S1 to human ACE2 cells was detected by Attune flow cytometer, then plotted in Prism 8.1 software. IC50 was calculated. FIG. 15 shows examples of IgG antibody neutralization of S1 protein binding to human ACE2/CHOK1 cells in a FACS assay.

The IC50 values are shown in the table below.

TABLE 16

IC₅₀ Values of Exemplary Antibodies for Inhibiting S1 protein Binding to ACE2/CHO-K1

| S1-ACE2/CHO-K1 | 2020EP54-E10 | 2020EP054-E12 | hACE2-Fc |
|---|---|---|---|
| IC₅₀ (nM) | 2.712 | 1.90 | 5.23 |

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. An isolated antibody that binds a S1 subunit of a SARS-CoV-2 spike protein, wherein the antibody binds to the same epitope as a reference antibody or competes against the reference antibody from binding the S1 subunit, and wherein the reference antibody is selected from the group consisting of 2020EP53-D06, 2020EP54-H01, 2020EP54-E12, 2020EP54-B12, 2020EP54-B02, 2020EP54-E10, 2020EP60-F05, 2020EP60-A12, 2020EP61-A08, 2020EP61-C12, 2020EP64-G10, 2020EP66-D03, 2020EP64-C08, 2020EP66-A07, 2020EP71-E04, and 2020EP75-E02.

2. The isolated antibody of claim 1, wherein the epitope is located in a receptor binding domain (RBD) of the S1 subunit.

3. The isolated antibody of claim 1, wherein the epitope is located outside of a RBD of the S1 subunit.

4. The isolated antibody of claim 1, wherein the antibody comprises:
(a) a heavy chain complementary determining region 1 (HC CDR1), a heavy chain complementary determining region 2 (HC CDR2), and a heavy chain complementary determining region 3 (HC CDR3), wherein the HC CDR1, HC CDR2, and HC CDR3 collectively are at least 80% identical to the heavy chain CDRs of the reference antibody; and/or
(b) a light chain complementary determining region 1 (LC CDR1), a light chain complementary determining region 2 (LC CDR2), and a light chain complementary determining region 3 (LC CDR3), wherein the LC CDR1, LC CDR2, and LC CDR3 collectively are at least 80% identical to the light chain CDRs of the reference antibody.

5. The isolated antibody of claim 1, wherein the HC CDRs of the antibody collectively contain no more than 8 amino acid residue variations as compared with the HC CDRs of the reference antibody; and/or wherein the LC CDRs of the antibody collectively contain no more than 8 amino acid residue variations as compared with the LC CDRs of the reference antibody.

6. The isolated antibody of claim 1, wherein the antibody comprises a V_{H} that is at least 85% identical to the V_{H} of the reference antibody, and/or a V_{L} that is at least 85% identical to the V_{L} of the reference antibody.

7. The isolated antibody of claim 1, wherein the antibody has a binding affinity of less than 10 nM to S1.

8. The isolated antibody of claim 1, which comprises the same heavy chain complementary determining regions (HC CDRs) and the same light chain complementary determining regions (LC CDRs) as the reference antibody.

9. The isolated antibody of claim 8, which comprises the same V_{H} and the same V_{L} as the reference antibody.

10. The isolated antibody of claim 1, wherein the antibody is a human antibody or a humanized antibody.

11. The isolated antibody of claim 1, wherein the antibody is a full-length antibody or an antigen-binding fragment thereof.

12. The isolated antibody of claim 1, wherein the antibody is a single-chain antibody (scFv).

13. The isolated antibody of claim 11, wherein the antibody is a full-length antibody, which is an IgG1 molecule.

14. A nucleic acid or a set of nucleic acids, which collectively encode the antibody of claim 1.

15. The nucleic acid or the set of nucleic acids of claim 14, which is a vector or a set of vectors.

16. The nucleic acid or the set of nucleic acids or claim 15, wherein the vector is an expression vector.

17. A host cell comprising the nucleic acid or the set of nucleic acids of claim 14.

18. A pharmaceutical composition comprising the antibody of claim 1, or the nucleic acid or nucleic acids encoding same,, and a pharmaceutically acceptable carrier.

19. A method of treating or inhibiting a coronavirus infection in a subject, comprising administering to a subject in need thereof an effective amount of the pharmaceutical composition of claim 18.

20. The method of claim 19, wherein the coronavirus is selected from the group consisting of severe acute respiratory syndrome coronavirus 2 (SARS-CoV-2), severe acute respiratory syndrome coronavirus (SARS-CoV), and Middle East respiratory syndrome coronavirus (MERS-CoV).

21. The method of claim 20, wherein the coronavirus is SARS-CoV-2.

22. The method of claim 19, wherein the subject has, is suspected of having, or is at risk of having, a disease selected from the group consisting of COVID-19, SARS, and MERS.

23. The method of claim 22, wherein the disease is COVID-19.

24. The method of claim 19 wherein the subject is a human patient.

25. A method of detecting presence of SARS-CoV-2, comprising:
(i) contacting an antibody of claim 1 with a sample suspected of containing an S1 protein of a SARS-CoV-2 virus, or a fragment comprising a RBD thereof; and,
(ii) detecting binding of the antibody to the S1 protein or the fragment comprising the RBD.

26. The method of claim 25, wherein the antibody is conjugated to a detectable label.

27. The method of claim 25 , wherein the sample is a biological sample obtained from a subject suspected of having a SARS-CoV-2 infection.

28. The method of claim 27, wherein the biological sample is a blood sample.

29. A method of producing an antibody binding to a S1 subunit of a SARS-CoV-2 spike protein, comprising:
(i) culturing the host cell of claim 17 under conditions allowing for expression of the antibody that binds to the S1 subunit; and,
(ii) harvesting the antibody thus produced from the cell culture.


SEQUENCE LISTING (50 sequences)

SEQ ID NO: 1 - protein, 123 aa, Artificial Sequence
  Synthetic
QVQLVQSGGGVVQPGRSLRLSCAASGFTFSSYAMHWVRQAPGKGLEWVAVISYDGSNKYYADSVKGRFTISRDNSKNTLYLQMNSLRAEDTAVYYCARDGEDGYNYISPFDYWGQGTLVTVSS

SEQ ID NO: 2 - protein, 105 aa, Artificial Sequence
  Synthetic
SYELTQPPSVSVAPGETARITCRGNDIGSKSVHWYQQKPGQAPVLVLYYDSDRPSGIPERFSGSNSGNTATLTISSVEAGDEADYYCQVWDIDVVFGGGTKLTVL

SEQ ID NO: 3 - protein, 117 aa, Artificial Sequence
  Synthetic
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAKGRGWYLDYWGQGTLVTVSS

SEQ ID NO: 4 - protein, 108 aa, Artificial Sequence
  Synthetic
NFMLTQPPSVSVSPGQTASITCSGDKLGDKYACWYQQKPGQSPVLVMYEDYKRPSGIPARFSGSNSGNTATLTISRVEGGDEGDYYCQVWDSRGPEVIFGGGTKLTVL

SEQ ID NO: 5 - protein, 120 aa, Artificial Sequence
  Synthetic
EVQLVESGGGLVQPGGSLRLSCAVTGFIVSSNYMNWVRQAPGKGLEWVSVIYSGGTTYYADSVKGRFTISRDKSKNTLYLQMNNLRAEDTAMYYCATLGNDYGDYGTDYWGQGTLVTVSS

SEQ ID NO: 6 - protein, 108 aa, Artificial Sequence
  Synthetic
QLVLTQPPSVSVSPGQTASISCSGDRLGQKYTSWYQQRPGQSPVLIMYQDNKRPSGIPERFSGSNSGNTATLTISGIQSMDEADYYCQVWDSGSDHAVFGGGTQLTVL

SEQ ID NO: 7 - protein, 117 aa, Artificial Sequence
  Synthetic
EVQLVESGGGLVQPGGSLRLSCAASGFAFSSYAMSWVRQAPGKGLEWVSAISGSGGSTYYADSVKGRFTISRDNSKNTLYLQMNSLRAEDTAVYYCAKVTRVGFDYWGQGTLVTVSS

SEQ ID NO: 8 - protein, 107 aa, Artificial Sequence
  Synthetic
SYELTQPPSVSVAPGKTARITCGGNNIGSKSVHWYQQKPGQAPVLVIYYDSDRPSGIPERFSGSNSGNTATLTISRVEAEDEADYYCQVWDSSSDVVFGGGTKLTVL

SEQ ID NO: 9 - protein, 119 aa, Artificial Sequence
  Synthetic
QVQLVQSGAEVKKPGASVKVSCKASGYTFIGYYMHWMRQAPGQGLEWMGRINPNSGGTNYAEKFQGRVTMTRDTSISTAYMELSRLTFNDTAVYYCARWDSSSWKFDSWGQGTLVTVSS

SEQ ID NO: 10 - protein, 107 aa, Artificial Sequence
  Synthetic
EIVLTQSPSSLSASVGDRVTITCQASQDISNYLNWYQQKPGKAPKLLIYDASNLETGVPSRFSGSGSGTDFTFTISSLQPEDIATYYCQQYDNLPITFGQGTRLEIK

SEQ ID NO: 11 - protein, 127 aa, Artificial Sequence
  Synthetic
QVQLVQSGAEVRKPGSSVKVSCKASGGTFSSFAISWVRQAPGQGLEWMGGIIPIFDTATYAQNFQGRVRMTADESTNTAYMELSSLTSEDTAVYYCARSTSYYDSRGDYKVGDFDYWGQGTLVTVSS

SEQ ID NO: 12 - protein, 107 aa, Artificial Sequence
  Synthetic
AIRMTQSPSSLSASVGDRVTITCQASQDISNYLNWYQQKPGKAPKLLIYDASNLETGVPSRFSGSGSGTDFTFTISSLQPEDIATYYCQQYDNLPLTFGGGTKVEIK

SEQ ID NO: 13 - protein, 127 aa, Artificial Sequence
  Synthetic
QVQLVESGGGVVQPGGSLRLSCAASGFTFSSYGMHWVRQAPGKGLEWVAVISYDGSNKYYADSVKGRFTISRDNSKNTLYLQMNSLRAEDTAVYYCAKARVALGYFDWLLYDGMDVWGQGTTVTVSS

SEQ ID NO: 14 - protein, 106 aa, Artificial Sequence
  Synthetic
SSELTQPPSVSVSPGQTASITCSGDKLGNKYVFWYQQKPGQSPILVIYQDNRRPSGIPERFSGSNSGNTATLTISGDPAMDEADYYCQAWDSSTVVFGGGTKLTVL

SEQ ID NO: 15 - protein, 117 aa, Artificial Sequence
  Synthetic
EVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAKGLEYYFDYWGQGTLVTVSS

SEQ ID NO: 16 - protein, 106 aa, Artificial Sequence
  Synthetic
SYELTQPPSLSVSPGQTARITCSGDALPEQYAYWYQQKPGQAPVLVIYKDSERPSGIPERFSGSSSGTTVTLTISGVQAEDEADYYCQAWDSSTVVFGGGTKLTVL

SEQ ID NO: 17 - protein, 125 aa, Artificial Sequence
  Synthetic
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARESGNYYYDSSGYTFDYWGQGTLVTVSS

SEQ ID NO: 18 - protein, 106 aa, Artificial Sequence
  Synthetic
SYVLTQPLSVSVALGQTARITCGGNNIGSKNVHWYQQKPGQAPVLVIYYDSDRPSGIPERFSGSNSGNTATLTISGTQAMDEADYYCQAWDSSTVVFGGGTKLTVL

SEQ ID NO: 19 - protein, 121 aa, Artificial Sequence
  Synthetic
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKDEYSYGSLYFDYWGQGTLVTVSS

SEQ ID NO: 20 - protein, 108 aa, Artificial Sequence
  Synthetic
SYELTQPPSVSVAPGQTARITCGGNNIGSKSVHWYQQKPGQAPVLVVYDDSDRPSGIPERFSGSNSGNTATLTISRVEAGDEADYYCQVWDSSSDSVVFGGGTKLTVL

SEQ ID NO: 21 - protein, 105 aa, Artificial Sequence
  Synthetic
SYELTQPPSVSVAPGETARITCRGNDIGSKSVHWYQQKPGQAPVLVLYYDSDRPSGIPERFSGSNSGNTATLTISSVEAGDEADYYCHVWDIDVVFGGGTKLTVL

SEQ ID NO: 22 - protein, 121 aa, Artificial Sequence
  Synthetic
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARGPPLWNYGKGFDYWGQGTLVTVSS

SEQ ID NO: 23 - protein, 110 aa, Artificial Sequence
  Synthetic
QPVLTQPPSASGTPGQRVTISCSGSSSNIGSNYVYWYQQLPRTAPKLLIYRNNQRPSGVPDRFSGSKSGTSASLAISGLRSEDEADYYCAAWDDSLSAWVFGGGTKLTVL

SEQ ID NO: 24 - protein, 116 aa, Artificial Sequence
  Synthetic
QMQLVQSGAEVKKPGSSVKVSCKASGGTFSSYAISWVRQAPRQGLEWMGRIIPILGIANYAQKFQGRVTITADKSTSTAYMELSSLRSEDTAVYYCASSYGGNQLWGQGTLVTVSS

SEQ ID NO: 25 - protein, 110 aa, Artificial Sequence
  Synthetic
QSVLTQPPSASGTPGQRVTISCSGSDSNIGTNTVNWYQQVPGTAPKVLIYSTHQRPSGVPDRFSASKSGTSASLAISGLQSEDEADYYCSSWDVSLNAWVFGGGTKVTVL

SEQ ID NO: 26 - protein, 125 aa, Artificial Sequence
  Synthetic
EVQLVQSGAEVKKPGSSVKVSCKASGGTFSSYAISWVRQAPGQGLEWMGGIIPIFGTANYAQKFQGRVTITADESTSTAYMELSSLRSEDTAVYYCASGGDGYNYDYYYYGMDVWGQGTTVTVSS

SEQ ID NO: 27 - protein, 106 aa, Artificial Sequence
  Synthetic
SYELTQPPSVSVSPGQTATITCSGEKLGDKYSFWYQQKPGQSPVMVMYQDDQRPSGTPERFSGSNSGNTATLTISGTRATDEADYYCQAWDNSASVFGSGTKLTVL

SEQ ID NO: 28 - protein, 126 aa, Artificial Sequence
  Synthetic
QVQLVQSGGGVVQPGRSLRLSCAASGFTFSSYGMHWVRQAPGKGLEWVAVISYDGSNKYYADSVKGRFTISRDNSKNTLYLQMNSLRAEDTAVYYCAKDRAYYYGYYYYYYGMDVWGQGTTVTVSS

SEQ ID NO: 29 - protein, 111 aa, Artificial Sequence
  Synthetic
QSVLTQPPSVSGAPGQRVTISCTGSSSNIGAGYDVHWYQQLPGTAPKLLIYGNSNRPSGVPDRFSGSKSGTSASLAITGLQAEDEADYYCATWDDSLSGPVFGGGTKVTVL

SEQ ID NO: 30 - protein, 121 aa, Artificial Sequence
  Synthetic
QMQLVQSGAEVKKPGSSVKVSCKASGGTFSSYAISWVRQAPGQGLEWMGGIIPIFGTANYAQKFQGRVTITADKSTSTAYMELSSLRSEDTAVYYCARPRDYRAQYYFDYWGQGTLVTVSS

SEQ ID NO: 31 - protein, 107 aa, Artificial Sequence
  Synthetic
DIVMTQSPSSLSASVGDRVTITCRAGQDIDTSVNWYQLKPGKAPRLLIYASSSLQTGVPSRFSGGGSGAEFTLTISSLQPEDFATYFCQQTFSTSVTFGGGTKVEIK

SEQ ID NO: 32 - protein, 243 aa, Artificial Sequence
  Synthetic
QVQLVQSGGGVVQPGRSLRLSCAASGFTFSSYAMHWVRQAPGKGLEWVAVISYDGSNKYYADSVKGRFTISRDNSKNTLYLQMNSLRAEDTAVYYCARDGEDGYNYISPFDYWGQGTLVTVSSGGGGSGGGGSGGGGSSYELTQPPSVSVAPGETARITCRGNDIGSKSVHWYQQKPGQAPVLVLYYDSDRPSGIPERFSGSNSGNTATLTISSVEAGDEADYYCQVWDIDVVFGGGTKLTVL

SEQ ID NO: 33 - protein, 240 aa, Artificial Sequence
  Synthetic
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAKGRGWYLDYWGQGTLVTVSSGGGGSGGGGSGGGGSNFMLTQPPSVSVSPGQTASITCSGDKLGDKYACWYQQKPGQSPVLVMYEDYKRPSGIPARFSGSNSGNTATLTISRVEGGDEGDYYCQVWDSRGPEVIFGGGTKLTVL

SEQ ID NO: 34 - protein, 243 aa, Artificial Sequence
  Synthetic
EVQLVESGGGLVQPGGSLRLSCAVTGFIVSSNYMNWVRQAPGKGLEWVSVIYSGGTTYYADSVKGRFTISRDKSKNTLYLQMNNLRAEDTAMYYCATLGNDYGDYGTDYWGQGTLVTVSSGGGGSGGGGSGGGGSQLVLTQPPSVSVSPGQTASISCSGDRLGQKYTSWYQQRPGQSPVLIMYQDNKRPSGIPERFSGSNSGNTATLTISGIQSMDEADYYCQVWDSGSDHAVFGGGTQLTVL

SEQ ID NO: 35 - protein, 239 aa, Artificial Sequence
  Synthetic
EVQLVESGGGLVQPGGSLRLSCAASGFAFSSYAMSWVRQAPGKGLEWVSAISGSGGSTYYADSVKGRFTISRDNSKNTLYLQMNSLRAEDTAVYYCAKVTRVGFDYWGQGTLVTVSSGGGGSGGGGSGGGGSSYELTQPPSVSVAPGKTARITCGGNNIGSKSVHWYQQKPGQAPVLVIYYDSDRPSGIPERFSGSNSGNTATLTISRVEAEDEADYYCQVWDSSSDVVFGGGTKLTVL

SEQ ID NO: 36 - protein, 241 aa, Artificial Sequence
  Synthetic
QVQLVQSGAEVKKPGASVKVSCKASGYTFIGYYMHWMRQAPGQGLEWMGRINPNSGGTNYAEKFQGRVTMTRDTSISTAYMELSRLTFNDTAVYYCARWDSSSWKFDSWGQGTLVTVSSGGGGSGGGGSGGGGSEIVLTQSPSSLSASVGDRVTITCQASQDISNYLNWYQQKPGKAPKLLIYDASNLETGVPSRFSGSGSGTDFTFTISSLQPEDIATYYCQQYDNLPITFGQGTRLEIK

SEQ ID NO: 37 - protein, 249 aa, Artificial Sequence
  Synthetic
QVQLVQSGAEVRKPGSSVKVSCKASGGTFSSFAISWVRQAPGQGLEWMGGIIPIFDTATYAQNFQGRVRMTADESTNTAYMELSSLTSEDTAVYYCARSTSYYDSRGDYKVGDFDYWGQGTLVTVSSGGGGSGGGGSGGGGSAIRMTQSPSSLSASVGDRVTITCQASQDISNYLNWYQQKPGKAPKLLIYDASNLETGVPSRFSGSGSGTDFTFTISSLQPEDIATYYCQQYDNLPLTFGGGTKVEIK

SEQ ID NO: 38 - protein, 248 aa, Artificial Sequence
  Synthetic
QVQLVESGGGVVQPGGSLRLSCAASGFTFSSYGMHWVRQAPGKGLEWVAVISYDGSNKYYADSVKGRFTISRDNSKNTLYLQMNSLRAEDTAVYYCAKARVALGYFDWLLYDGMDVWGQGTTVTVSSGGGGSGGGGSGGGGSSSELTQPPSVSVSPGQTASITCSGDKLGNKYVFWYQQKPGQSPILVIYQDNRRPSGIPERFSGSNSGNTATLTISGDPAMDEADYYCQAWDSSTVVFGGGTKLTVL

SEQ ID NO: 39 - protein, 238 aa, Artificial Sequence
  Synthetic
EVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAKGLEYYFDYWGQGTLVTVSSGGGGSGGGGSGGGGSSYELTQPPSLSVSPGQTARITCSGDALPEQYAYWYQQKPGQAPVLVIYKDSERPSGIPERFSGSSSGTTVTLTISGVQAEDEADYYCQAWDSSTVVFGGGTKLTVL

SEQ ID NO: 40 - protein, 246 aa, Artificial Sequence
  Synthetic
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARESGNYYYDSSGYTFDYWGQGTLVTVSSGGGGSGGGGSGGGGSSYVLTQPLSVSVALGQTARITCGGNNIGSKNVHWYQQKPGQAPVLVIYYDSDRPSGIPERFSGSNSGNTATLTISGTQAMDEADYYCQAWDSSTVVFGGGTKLTVL

SEQ ID NO: 41 - protein, 244 aa, Artificial Sequence
  Synthetic
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKDEYSYGSLYFDYWGQGTLVTVSSGGGGSGGGGSGGGGSSYELTQPPSVSVAPGQTARITCGGNNIGSKSVHWYQQKPGQAPVLVVYDDSDRPSGIPERFSGSNSGNTATLTISRVEAGDEADYYCQVWDSSSDSVVFGGGTKLTVL

SEQ ID NO: 42 - protein, 243 aa, Artificial Sequence
  Synthetic
QVQLVQSGGGVVQPGRSLRLSCAASGFTFSSYAMHWVRQAPGKGLEWVAVISYDGSNKYYADSVKGRFTISRDNSKNTLYLQMNSLRAEDTAVYYCARDGEDGYNYISPFDYWGQGTLVTVSSGGGGSGGGGSGGGRSSYELTQPPSVSVAPGETARITCRGNDIGSKSVHWYQQKPGQAPVLVLYYDSDRPSGIPERFSGSNSGNTATLTISSVEAGDEADYYCHVWDIDVVFGGGTKLTVL

SEQ ID NO: 43 - protein, 246 aa, Artificial Sequence
  Synthetic
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARGPPLWNYGKGFDYWGQGTLVTVSSGGGGSGKGGSGGGGSQPVLTQPPSASGTPGQRVTISCSGSSSNIGSNYVYWYQQLPRTAPKLLIYRNNQRPSGVPDRFSGSKSGTSASLAISGLRSEDEADYYCAAWDDSLSAWVFGGGTKLTVL

SEQ ID NO: 44 - protein, 241 aa, Artificial Sequence
  Synthetic
QMQLVQSGAEVKKPGSSVKVSCKASGGTFSSYAISWVRQAPRQGLEWMGRIIPILGIANYAQKFQGRVTITADKSTSTAYMELSSLRSEDTAVYYCASSYGGNQLWGQGTLVTVSSGGGGSGGGGSGGGGSQSVLTQPPSASGTPGQRVTISCSGSDSNIGTNTVNWYQQVPGTAPKVLIYSTHQRPSGVPDRFSASKSGTSASLAISGLQSEDEADYYCSSWDVSLNAWVFGGGTKVTVL

SEQ ID NO: 45 - protein, 246 aa, Artificial Sequence
  Synthetic
EVQLVQSGAEVKKPGSSVKVSCKASGGTFSSYAISWVRQAPGQGLEWMGGIIPIFGTANYAQKFQGRVTITADESTSTAYMELSSLRSEDTAVYYCASGGDGYNYDYYYYGMDVWGQGTTVTVSSGGGGSGGGGSGGGGSSYELTQPPSVSVSPGQTATITCSGEKLGDKYSFWYQQKPGQSPVMVMYQDDQRPSGTPERFSGSNSGNTATLTISGTRATDEADYYCQAWDNSASVFGSGTKLTVL

SEQ ID NO: 46 - protein, 252 aa, Artificial Sequence
  Synthetic
QVQLVQSGGGVVQPGRSLRLSCAASGFTFSSYGMHWVRQAPGKGLEWVAVISYDGSNKYYADSVKGRFTISRDNSKNTLYLQMNSLRAEDTAVYYCAKDRAYYYGYYYYYYGMDVWGQGTTVTVSSGGGGSGGGGSGGGGSQSVLTQPPSVSGAPGQRVTISCTGSSSNIGAGYDVHWYQQLPGTAPKLLIYGNSNRPSGVPDRFSGSKSGTSASLAITGLQAEDEADYYCATWDDSLSGPVFGGGTKVTVL

SEQ ID NO: 47 - protein, 243 aa, Artificial Sequence
  Synthetic
QMQLVQSGAEVKKPGSSVKVSCKASGGTFSSYAISWVRQAPGQGLEWMGGIIPIFGTANYAQKFQGRVTITADKSTSTAYMELSSLRSEDTAVYYCARPRDYRAQYYFDYWGQGTLVTVSSGGGGSGGGGSGGGGSDIVMTQSPSSLSASVGDRVTITCRAGQDIDTSVNWYQLKPGKAPRLLIYASSSLQTGVPSRFSGGGSGAEFTLTISSLQPEDFATYFCQQTFSTSVTFGGGTKVEIK

SEQ ID NO: 48 - protein, 1273 aa, Artificial Sequence
  Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 49 - protein, 433 aa, Artificial Sequence
  Synthetic
RFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 214 aa, Artificial Sequence
  Synthetic
RFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLGSHHHHHH